(19) United States
(12) Patent Application Publication
(10) Pub. No.: US 2003/0125236 A1
(43) Pub. Date: Jul. 3, 2003

(54) Particles for inhalation having rapid release properties

(75) Inventors: Jennifer L. Schmitke, Boston, MA (US); Donghao Chen, Lexington, MA (US); Richard P. Batycky, Newton, MA (US); David A. Edwards, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Advenced Inhalation Research, Inc., Cambridge, MA (US)

(21) Appl. No.: 10/179,463

(22) Filed: Jun. 24, 2002

Related U.S. Application Data
(63) Continuation-in-part of application No. 09/888,126, filed on Jun. 22, 2001.
Application No. 09/888,126 is a continuation-in-part of application No. 09/752,109, filed on Dec. 29, 2000, now abandoned.

(51) Int. Cl. A61K 38/28; A61K 31/19

(52) U.S. Cl. 514/3; 514/574

(57) ABSTRACT

The invention generally relates to formulations having particles comprising phospholipids, bioactive agent and excipients and the pulmonary delivery thereof. Dry powder inhaled insulin formulations are disclosed. Improved formulations comprising DPPC, insulin and sodium citrate which are useful in the treatment of diabetes are disclosed. Also, the invention relates to a method of for the pulmonary delivery of a bioactive agent comprising administering to the respiratory tract of a patient in need of treatment, or diagnosis an effective amount of particles comprising a bioactive agent or any combination thereof in association, wherein release of the agent from the administered particles occurs in a rapid fashion.

### RELATED APPLICATIONS

This application is a continuation-in-part of and claims priority to U.S. application Ser. No. 09/888,126 filed on Jun. 22, 2001, which is a continuation-in-part of and claims priority to U.S. application Ser. No. 09/752,109 filed on Dec. 29, 2000. The entire teachings of the above applications are incorporated herein by reference.

### BACKGROUND OF THE INVENTION

Pulmonary delivery of bioactive agents, for example, therapeutic, diagnostic and prophylactic agents provides an attractive alternative to, for example, oral, transdermal and parenteral administration. That is, pulmonary administration can typically be completed without the need for medical intervention (self-administration), the pain often associated with injection therapy is avoided, and the amount of enzymatic and pH mediated degradation of the bioactive agent, frequently encountered with oral therapies, can be significantly reduced. In addition, the lungs provide a large mucosal surface for drug absorption and there is no first-pass liver effect of absorbed drugs. Further, it has been shown that high bioavailability of many molecules, for example, macromolecules, can be achieved via pulmonary delivery or inhalation. Typically, the deep lung, or alveoli, is the primary target of inhaled bioactive agents, particularly for agents requiring systemic delivery.

The release kinetics or release profile of a bioactive agent into the local and/or systemic circulation is a key consideration in most therapies, including those employing pulmonary delivery. That is, many illnesses or conditions require administration of a constant or sustained level of a bioactive agent to provide an effective therapy. Typically, this can be accomplished through a multiple dosing regimen or by employing a system that releases the medicament in a sustained fashion.

Delivery of bioactive agents to the pulmonary system, however, can result in rapid release of the agent following administration. For example, U.S. Pat. No. 5,997,848 to Patton et al. describes the absorption of insulin following administration of a dry powder formulation via pulmonary delivery. The peak insulin level was reached in about 30 minutes for primates and in about 20 minutes for human subjects. Further, Heinemann, Traut and Heise teach in Diabetic Medicine (14:63-72 (1997)) that the onset of action after inhalation reached half-maximal action in about 30 minutes, assessed by glucose infusion rate in healthy volunteers.

Diabetes mellitus is the most common of the serious metabolic diseases affecting humans. It may be defined as a state of chronic hyperglycaemia, i.e., excess sugar in the blood, that results from a relative or absolute lack of insulin action. Insulin is a peptide hormone produced and secreted by B cells within the islets of Langerhans in the pancreas. Insulin promotes glucose utilization, protein synthesis, and the formation and storage of neutral lipids. It is generally required for the entry of glucose into muscle. Glucose, or “blood sugar,” is the principal source of carbohydrate energy for man and many other organisms. Excess glucose is stored in the body as glycogen, which is metabolized into glucose as needed to meet bodily requirements.

The hyperglycaemia associated with diabetes mellitus is a consequence of both the underutilization of glucose and the overproduction of glucose from protein due to relatively depressed or nonexistent levels of insulin. Diabetic patients frequently require daily, usually multiple, injections of insulin that may cause discomfort. This discomfort leads many type 2 diabetic patients to refuse to use insulin injections, even when they are indicated.

A need exists for formulations suitable for efficient inhalation comprising bioactive agents, for example, insulin, and wherein the bioactive agent of the formulation is released in a manner that is at least as efficient as presently available treatments and prophylactics, especially for the treatment of diabetes.

A need also exists for formulations suitable for delivery to the lung and rapid release into the systemic and/or local circulation. Such formulations are expected to increase the willingness of patients to comply with prescribed therapy, and may achieve improved disease treatment and control.

### SUMMARY OF THE INVENTION

Formulations having particles comprising, by weight, approximately 40% to approximately 60% DPPC, approximately 30% to approximately 50% insulin and approximately 10% sodium citrate are disclosed. In one embodiment, the particles comprise, by weight, 40% to 60% DPPC, 30% to 50% insulin and 10% sodium citrate. In another embodiment, the particle comprise, by weight, 40% DPPC, 50% insulin and 10% sodium citrate. In yet another embodiment, the particles comprise, by weight, 60% DPPC, 30% insulin and 10% sodium citrate.

Formulations having particles comprising, by weight, approximately 75% to approximately 80% DPPC, approximately 10% to approximately 15% insulin and approximately 10% sodium citrate are also disclosed. In one embodiment, the particles comprise, by weight, 75% to 80% DPPC, 10% to 15% insulin and 10% sodium citrate. In another embodiment, the particles comprise, by weight, 75% DPPC, 15% insulin and 10% sodium citrate. In yet another embodiment, the particles comprise, by weight, 80% DPPC, 10% insulin and 10% sodium citrate.

The present invention also features methods for treating a human patient in need of insulin comprising administering pulmonarily to the respiratory tract of a patient in need of treatment, an effective amount of particles comprising by weight, approximately 40% to approximately 60% DPPC, approximately 30% to approximately 50% insulin and approximately 10% sodium citrate, wherein release of the insulin is rapid. In one embodiment, the particles comprise, by weight, 40% to 60% DPPC, 30% to 50% insulin and 10% sodium citrate. In another embodiment, the particle comprise, by weight, 40% DPPC, 50% insulin and 10% sodium citrate. In yet another embodiment, the particles comprise, by weight, 60% DPPC, 30% insulin and 10% sodium citrate. This method is particularly useful for the treatment of diabetes. If desired, the particles can be delivered in a single, breath actuated step.

The present invention also features methods for treating a human patient in need of insulin comprising administering pulmonarily to the respiratory tract of a patient in need of treatment, an effective amount of particles comprising by weight, approximately 75% to approximately 80% DPPC, approximately 10% to approximately 15% insulin and approximately 10% sodium citrate, wherein release of the insulin is rapid. In one embodiment, the particles comprise, by weight, 75% to 80% DPPC, 10% to 15% insulin and 10% sodium citrate. In another embodiment, the particle comprise, by weight, 75% DPPC, 15% insulin and 10% sodium citrate. In yet another embodiment, the particles comprise, by weight, 80% DPPC, 10% insulin and 10% sodium citrate This method is particularly useful for the treatment of diabetes. If desired, the particles can be delivered in a single, breath actuated step.

In addition, the present invention features methods of delivering an effective amount of insulin to the pulmonary system, comprising providing a mass of particles comprising by weight, approximately 40% to approximately 60% DPPC, approximately 30% to approximately 50% insulin and approximately 10% sodium citrate; and administering via simultaneous dispersion and inhalation the particles, from a receptacle having the mass of the particles, to a human subject's respiratory tract, wherein release of the insulin is rapid. Particularly useful for rapid release are formulations comprising low transition temperature phospholipids. In one embodiment, the particles comprise, by weight, 40% to 60% DPPC, 30% to 50% insulin and 10% sodium citrate. In another embodiment, the particles comprise, by weight, 40% DPPC, 50% insulin and 10% sodium citrate. In yet another embodiment, the particles comprise, by weight, 60% DPPC, 30% insulin and 10% sodium citrate.

The present invention also features methods of delivering an effective amount of insulin to the pulmonary system, comprising providing a mass of particles comprising by weight, approximately 75% to approximately 80% DPPC, approximately 10% to approximately 15% insulin and approximately 10% sodium citrate; and administering via simultaneous dispersion and inhalation the particles, from a receptacle having the mass of the particles, to a human subject's respiratory tract, wherein release of the insulin is rapid. Particularly useful for rapid release are formulations comprising low transition temperature phospholipids. In one embodiment, the particles comprise, by weight, 75% to 80% DPPC, 10% to 15% insulin and 10% sodium citrate. In another embodiment, the particles comprise, by weight, 75% DPPC, 15% insulin and 10% sodium citrate. In yet another embodiment, the particles comprise, by weight, 80% DPPC, 10% insulin and 10% sodium citrate.

The invention also features a kit comprising two or more receptacles comprising unit dosages selected from the insulin formulations described herein. For example, the formulation can be particles comprising, by weight, approximately 60% DPPC, approximately 30% insulin and approximately 10% sodium citrate; or comprising, by weight, approximately 40% DPPC, approximately 50% insulin and approximately 10% sodium citrate; or comprising, by weight, approximately 40% to approximately 60% DPPC, approximately 30% to approximately 50% insulin and approximately 10% sodium citrate or comprising by weight, approximately 80% DPPC, approximately 10% insulin and approximately 10% sodium citrate; or comprising, by weight, approximately 75% to approximately 80% DPPC, approximately 10% to approximately 15% insulin and approximately 10% sodium citrate. In one embodiment, the receptacles contain particles having a formulation of 60% DPPC, 30% insulin and 10% sodium citrate; or comprising, by weight, 40% DPPC, 50% insulin and 10% sodium citrate; or comprising, by weight, 40% to 60% DPPC, 30% to 50% insulin and 10% sodium citrate or comprising by weight, 80% DPPC, 10% insulin and 10% sodium citrate; or comprising, by weight, 75% to 80% DPPC, 10% to 15% insulin and 10% sodium citrate. Combinations of receptacles containing different formulations within the same kit are also a feature of the present invention. For example, the kit can comprise two or more receptacles comprising unit dosages of particles comprising 40% to 60% DPPC, 30% to 50% insulin and 10% sodium citrate and one or more receptacles comprising unit dosages of particles comprising, by weight, 75% to 80% DPPC, 10% to 15% insulin and 10% sodium citrate. In another embodiment, the kit comprises one or more receptacles comprising unit dosages of particles comprising 60% DPPC, 30% insulin and 10% sodium citrate and one or more receptacles comprising unit dosages of particles comprising, by weight, 80% DPPC, 10% insulin and 10% sodium citrate. In another embodiment, the kit comprises one or more receptacles comprising a formulation of particles comprising 60% DPPC, 30% insulin and 10% sodium citrate and one or more receptacles comprising unit dosages of particles comprising, by weight, 75% DPPC, 15% insulin and 10% sodium citrate.

The present invention also features a kit comprising at least two receptacles each receptacle containing a different amount of dry powder insulin suitable for inhalation.

In another aspect, the invention features a formulation having particles comprising, by weight, 60% DPPC, 30% insulin and 10% sodium citrate, wherein the method of preparing the formulation comprises preparing a solution of DPPC; preparing a solution of insulin and sodium citrate; heating each of the solutions to a temperature of 50° C.; combining the two solutions such that the total solute concentration is greater than 3 grams per liter (e.g., 5, 10, or 15 grams/liter); and spray drying the combined solution to form particles. In one embodiment, the solute concentration of the combined solution is 15 grams per liter.

In still another aspect, the invention features a formulation having particles comprising, by weight, 75% DPPC, 15% insulin and 10% sodium citrate, wherein the method of preparing the formulation comprises preparing a solution of DPPC; preparing a solution of insulin and sodium citrate; heating each of the solutions to a temperature of 50° C.; combining the two solutions such that the total solute concentration is greater than 3 grams per liter (e.g., 5, 10, or 15 grams/liter); and spray drying the combined solution to form particles. In one embodiment, the solute concentration of the combined solution is 15 grams per liter.

In still another aspect, the invention features a formulation having particles comprising, by weight, 40% DPPC, 50% insulin and 10% sodium citrate, wherein the method of preparing the formulation comprises preparing a solution of DPPC; preparing a solution of insulin and sodium citrate; heating each of the solutions to a temperature of 50° C.; combining the two solutions such that the total solute concentration is greater than 3 grams per liter (e.g., 5, 10, or 15 grams/liter); and spray drying the combined solution to form particles. In one embodiment, the solute concentration of the combined solution is 15 grams per liter.

In another embodiment, the above-described particles comprise a mass of from about 1.5 mg to about 20 mg of insulin (for example, 1.0, 1.5, 2.5, 5, 7.5, 10, 12.5, 15, 17.5, 20, or 25 mg). In another embodiment, the dosage of insulin of any of the above particles is between about 42 IU and about 540 IU. Another effective dose for treatment of humans is between about 155 IU and about 170 IU. In another embodiment, the above-described particles have a tap density less than about 0.4 g/cm³ and/or a median geometric diameter of from between about 5 micrometers and about 30 micrometers and/or an aerodynamic diameter of from about 1 micrometer to about 5 micrometers.

The invention has numerous advantages. For example, particles suitable for inhalation can be designed to possess a controllable, in particular a rapid, release profile. This rapid release profile provides for abbreviated residence of the administered bioactive agent, in particular insulin, in the lung and decreases the amount of time in which therapeutic levels of the agent are present in the local environment or systemic circulation. The rapid release of agent provides a desirable alternative to injection therapy currently used for many therapeutic, diagnostic and prophylactic agents requiring rapid release of the agent, such as insulin for the treatment of diabetes. In addition, the invention provides a method of delivery to the pulmonary system wherein the high initial release of agent typically seen in inhalation therapy is boosted, giving very high initial release. Consequently, patient compliance and comfort can be increased by not only reducing frequency of dosing, but by providing a therapy that is more amenable to patients.

This dry powder delivery system allows for efficient dose delivery from a small, convenient and inexpensive delivery device. In addition, the simple and convenient inhaler together with the room temperature stable powder may offer an attractive replacement for currently available injections. This system has the potential to help achieve improved glycaemic control in patients with diabetes by increasing the willingness of patients to comply with insulin therapy.

### BRIEF DESCRIPTION Or THE DRAWINGS

FIG. 1 is a graph of the glucose infusion rate (GIR) over time for subjects administered inhaled insulin. In this graph, the pharmacodynamic profile of subjects administered 84 IU of inhaled insulin is identified by an open square; the pharmacodynamic profile of subjects administered 168 IU of inhaled insulin is identified by a closed square, and the pharmacodynamic profile of subjects administered 294 IU of inhaled insulin is identified by an open circle.

FIG. 2 is a graph of the glucose infusion rate (GIR) over time for subjects administered inhaled insulin (168 IU), subcutaneous insulin lispro (IL; 15 IU), or subcutaneous regular soluble insulin (RI; 15 IU). In this graph, the pharmacodynamic profile of subjects administered 15 IU of lispro is identified by an open triangle; the pharmacodynamic profile of subjects administered 15 IU of regular soluble insulin is identified by a closed triangle; and the pharmacodynamic profile of subjects administered 168 U of inhaled insulin is identified by a closed square.

FIG. 3 is a bar graph showing the onset of action, measured as the time to early 50% GIRₘₐₓ (in minutes) of inhaled insulin (AI; 84 IU, 168 IU, or 294 IU), lispro (IL; 15 IU), or regular soluble insulin (RI; 15 IU).

FIG. 4 is a bar graph of the GIR-AUC_{0-3 hours} for inhaled insulin (84 IU), insulin lispro (IL;15 IU), or regular soluble insulin (RI; 15 IU).

FIG. 5 is a bar graph of the biopotency of inhaled insulin (84 IU), expressed as a percent of the biopotency of insulin lispro (IL;15 IU) or regular soluble insulin (RI; 15 IU) during the first three or ten hours of administration.

FIG. 6 is a bar graph of the GIR-AUC evaluated as a function of time for inhaled insulin (AI; 84 IU, 168 IU, or 294 IU), insulin lispro (IL;15 IU), or regular soluble insulin (RI; 15 IU) with each data point represents individual dosing.

FIG. 7 is a graph of a dose-response over a range of doses for inhaled insulin (AI; 84 IU, 168 IU, or 294 IU).

The foregoing and other objects, features and advantages of the invention will be apparent from the following more particular description of preferred embodiments of the invention, as illustrated in the accompanying drawings.

### DETAILED DESCRIPTION OF THE INVENTION

The invention relates to particles capable of releasing bioactive agent, in particular insulin, in a rapid fashion. Methods of treating disease and delivery via the pulmonary system using these particles is also disclosed. As such, the particles possess rapid release properties. “Rapid release,” as that term is used herein, refers to an increased pharmacodynamic response (including, but not limited to serum levels of the bioactive agent and glucose infusion rates) typically seen in the first two hours following administration, and more preferably in the first hour. Rapid release also refers to a release of active agent, in particular inhaled insulin, in which the period of release of an effective level of agent is at least the same as, preferably shorter than that seen with presently available subcutaneous injections of active agent, in particular, insulin lispro and regular soluble insulin.

In one embodiment, the rapid release particles are formulated using insulin, sodium citrate and a phospholipid. It is believed that the selection of the appropriate phospholipid affects the release profile as described in more detail below. In a preferred embodiment, the rapid release is characterized by both the period of release being shorter and the levels of agent released being greater.

The particles of the invention have specific drug release properties. Release rates can be controlled as described below and as further described in U.S. application Ser. No. 09/644,736 filed Aug. 23, 2000 entitled “Modulation of Release From Dry Powder Formulations” by Sujit Basu, et al.

Drug release rates can be described in terms of the half-time of release of a bioactive agent from a formulation. As used herein the term “half-time” refers to the time required to release 50% of the initial drug payload contained in the particles. Fast or rapid drug release rates generally are less than 30 minutes and range from about 1 minute to about 60 minutes.

Drug release rates can also be described in terms of release constants. The first order release constant can be expressed using one of the following equations:

M_{pw(t)}=M₍ₒₒ₎*e^{-k*t} (1)

or,

M₍ₜ₎=M₍ₒₒ₎*(1−e^{-k*t}) (2)

Where k is the first order release constant. M₍ₒₒ₎ is the total mass of drug in the drug delivery system, e.g. the dry powder, and M_{pw(t)} is drug mass remaining in the dry powders at time t. M₍ₜ₎ is the amount of drug mass released from dry powders at time t. The relationship can be expressed as:

M₍ₒₒ₎=M_{pw(t)}+M₍ₜ₎ (3)

Equations (1), (2) and (3) may be expressed either in amount (i.e., mass) of drug released or concentration of drug released in a specified volume of release medium.

For example, Equation (2) may be expressed as:

C₍ₜ₎=C₍ₒₒ₎*(1−e^{-k*t}) (4)

Where k is the first order release constant. C₍ₒₒ₎ is the maximum theoretical concentration of drug in the release medium, and C₍ₜ₎ is the concentration of drug being released from dry powders to the release medium at time t.

The ‘half-time’ or t₅₀% for a first order release kinetics is given by a well-known equation,

t_{50%}=0.693/k (5)

Drug release rates in terms of first order release constant and t₅₀% may be calculated using the following equations:

k=−1n(M_{pw(t)}/M₍ₒₒ₎)/t (6)

or,

k=−1n(M₍ₒₒ₎−M₍ₜ₎)/M₍ₒₒ₎/t (7)

Release rates of drugs from particles can be controlled or optimized by adjusting the thermal properties or physical state transitions of the particles. The particles of the invention can be characterized by their matrix transition temperature. As used herein, the term “matrix transition temperature” refers to the temperature at which particles are transformed from glassy or rigid phase with less molecular mobility to a more amorphous, rubbery or molten state or fluid-like phase. As used herein, “matrix transition temperature” is the temperature at which the structural integrity of a particle is diminished in a manner which imparts faster release of drug from the particle. Above the matrix transition temperature, the particle structure changes so that mobility of the drug molecules increases resulting in faster release. In contrast, below the matrix transition temperature, the mobility of the drug particles is limited, resulting in a slower release. The “matrix transition temperature” can relate to different phase transition temperatures, for example, melting temperature (Tₘ), crystallization temperature (T_{c}) and glass transition temperature (T_{g}) which represent changes of order and/or molecular mobility within solids. The term “matrix transition temperature,” as used herein, refers to the composite or main transition temperature of the particle matrix above which release of drug is faster than below.

Experimentally, matrix transition temperatures can be determined by methods known in the art, in particular by differential scanning calorimetry (DSC). Other techniques to characterize the matrix transition behavior of particles or dry powders include synchrotron X-ray diffraction and freeze fracture electron microscopy.

Matrix transition temperatures can be employed to fabricate particles having desired drug release kinetics and to optimize particle formulations for a desired drug release rate. Particles having a specified matrix transition temperature can be prepared and tested for drug release properties by in vitro or in vivo release assays, pharmacokinetic studies and other techniques known in the art. Once a relationship between matrix transition temperatures and drug release rates is established, desired or targeted release rates can be obtained by forming and delivering particles which have the corresponding matrix transition temperature. Drug release rates can be modified or optimized by adjusting the matrix transition temperature of the particles being administered.

The particles of the invention include one or more materials which, alone or in combination, promote or impart to the particles a matrix transition temperature that yields a desired or targeted drug release rate. Properties and examples of suitable materials or combinations thereof are further described below. For example, to obtain a rapid release of a drug, materials, which, when combined, result in low matrix transition temperatures, are preferred. As used herein, “low transition temperature” refers to particles which have a matrix transition temperature which is below or about the physiological temperature of a subject. Particles possessing low transition temperatures tend to have limited structural integrity and be more amorphous, rubbery, in a molten state, or fluid-like.

Without wishing to be held to any particular interpretation of a mechanism of action, it is believed that, for particles having low matrix transition temperatures, the integrity of the particle matrix undergoes transition within a short period of time when exposed to body temperature (typically around 37° C.) and high humidity (approaching 100% in the lungs) and that the components of these particles tend to possess high molecular mobility allowing the drug to be quickly released and available for uptake. Designing and fabricating particles with a mixture of materials having high phase transition temperatures can be employed to modulate or adjust matrix transition temperatures of resulting particles and corresponding release profiles for a given drug.

Combining appropriate amounts of materials to produce particles having a desired transition temperature can be determined experimentally, for example, by forming particles having varying proportions of the desired materials, measuring the matrix transition temperatures of the mixtures (for example, by DSC), selecting the combination having the desired matrix transition temperature and, optionally, further optimizing the proportions of the materials employed.

Miscibility of the materials in one another also can be considered. Materials which are miscible in one another tend to yield an intermediate overall matrix transition temperature, all other things being equal. On the other hand, materials which are immiscible in one another tend to yield an overall matrix transition temperature that is governed either predominantly by one component or may result in biphasic release properties.

In a preferred embodiment, the particles include one or more phospholipids. The phospholipid or combination of phospholipids is selected to impart specific drug release properties to the particles. Phospholipids suitable for pulmonary delivery to a human subject are preferred. In one embodiment, the phospholipid is endogenous to the lung. In another embodiment, the phospholipid is non-endogenous to the lung.

The phospholipid can be present in the particles in an amount ranging from about 1 to about 99 weight %. Preferably, it can be present in the particles in an amount ranging from about 10 to about 80 weight %. In other example, the amount of phospholipid in the particles is approximately 40% to 80%, for example, 35%, 40%, 45%, 50%, 55%, 60%, 65%, 70%, 75%, 80% or 85%. In another example, the phospholipid is DPPC.

Examples of phospholipids include, but are not limited to, phosphatidic acids, phosphatidylcholines, phosphatidylethanolamines, phosphatidylglycerols, phosphatidylserines, phosphatidylinositols or a combination thereof. Modified phospholipids, for example, phospholipids having their head group modified, e.g., alkylated or polyethylene glycol (PEG)-modified, also can be employed.

In a preferred embodiment, the matrix transition temperature of the particles is related to the phase transition temperature, as defined by the melting temperature (Tₘ), the crystallization temperature (T_{c}) and the glass transition temperature (T_{g}) of the phospholipid or combination of phospholipids employed in forming the particles. Tₘ, T_{c} and T_{g} are terms known in the art. For example, these terms are discussed in Phospholipid Handbook (Gregor Cevc, editor, 1993, Marcel-Dekker, Inc.).

Phase transition temperatures for phospholipids or combinations thereof can be obtained from the literature. Sources listing phase transition temperatures of phospholipids include, for instance, the Avanti Polar Lipids (Alabaster, Ala.) Catalog or the Phospholipid Handbook (Gregor Cevc, editor, 1993, Marcel-Dekker, Inc.). Small variations in transition temperature values listed from one source to another may be the result of experimental conditions such as moisture content.

Experimentally, phase transition temperatures can be determined by methods known in the art, in particular by differential scanning calorimetry. Other techniques to characterize the phase behavior of phospholipids or combinations thereof include synchrotron X-ray diffraction and freeze fracture electron microscopy.

Combining the appropriate amounts of two or more phospholipids to form a combination having a desired phase transition temperature is described, for example, in the Phospholipid Handbook (Gregor Cevc, editor, 1993, Marcell-Dekker, Inc.). Miscibilities of phospholipids in one another may be found in the Avanti Polar Lipids (Alabaster, Ala.) Catalog.

The amounts of phospholipids to be used to form particles having a desired or targeted matrix transition temperature can be determined experimentally, for example, by forming mixtures in various proportions of the phospholipids of interest, measuring the transition temperature for each mixture, and selecting the mixture having the targeted transition temperature. The effects of phospholipid miscibility on the matrix transition temperature of the phospholipid mixture can be determined by combining a first phospholipid with other phospholipids having varying miscibilities with the first phospholipid and measuring the transition temperature of the combinations.

Combinations of one or more phospholipids with other materials also can be employed to achieve a desired matrix transition temperature. Examples include polymers and other biomaterials, such as, for instance, lipids, sphingolipids, cholesterol, surfactants, polyaminoacids, polysaccharides, proteins, salts and others. Amounts and miscibility parameters selected to obtain a desired or targeted matrix transition temperatures can be determined as described above.

In general, phospholipids, combinations of phospholipids, as well as combinations of phospholipids with other materials, which yield a matrix transition temperature no greater than about the physiological body temperature of a patient, are preferred in fabricating particles which have fast drug release properties. Such phospholipids or phospholipid combinations are referred to herein as having low transition temperatures. Examples of suitable low transition temperature phospholipids are listed in Table 1. Transition temperatures shown are obtained from the Avanti Polar Lipids (Alabaster, Ala.) Catalog.

TABLE 1

| | | Transition |
|---|---|---|
| | | Tem- |
| | Phospholipids | perature |
| 1 | 1,2-Dilauroyl-sn-glycero-3-phosphocholine (DLPC) | −1° C. |
| 2 | 1,2-Ditridecanoyl-sn-glycero-3-phosphocholine | 14° C. |
| 3 | 1,2-Dimyristoyl-sn-glycero-3-phosphocholine (DMPCQ) | 23° C. |
| 4 | 1,2-Dipentadecanoyl-sn-glycero-3-phosphocholine | 33° C. |
| 5 | 1,2-Dipalmitoyl-sn-glycero-3-phosphocholine (DPPC) | 41° C. |
| 6 | 1-Myristoyl-2-palmitoyl-sn-glycero-3-phosphocholine | 35° C. |
| 7 | 1-Myristoyl-2-stearoyl-sn-glycero-3-phosphocholine | 40° C. |
| 8 | 1-Palmitoyl-2-myristoyl-sn-glycero-3-phosphocholine | 27° C. |
| 9 | 1-Stearoyl-2-myristoyl-sn-glycero-3-phosphocholine | 30° C. |
| 10 | 1,2-Dilauroyl-sn-glycero-3-phosphate (DLPA) | 31° C. |
| 11 | 1,2-Dimyristoyl-sn-glycero-3-[phospho-L-serine] | 35° C. |
| 12 | 1,2-Dimyristoyl-sn-glycero-3-[phospho-rac-(1-glycerol)] | 23° C. |
| | (DMPG) | |
| 13 | 1,2-Dipalmitoyl-sn-glycero-3-[phospho-rac-(1-glycerol)] | 41° C. |
| | (DPPG) | |
| 14 | 1,2-Dilauroyl-sn-glycero-3-phosphoethanolamine (DLPE) | 29° C. |

Phospholipids having a head group selected from those found endogenously in the lung, e.g., phosphatidylcholine, phosphatidylethanolamines, phosphatidylglycerols, phosphatidylserines, phosphatidylinositols or a combination thereof are preferred.

The above materials can be used alone or in combinations. Other phospholipids which have a phase transition temperature no greater than a patient's body temperature, also can be employed, either alone or in combination with other phospholipids or materials.

The particles of the instant invention, in particular the rapid release particles, are delivered pulmonarily. “Pulmonary delivery,” as that term is used herein refers to delivery to the respiratory tract. The “respiratory tract,” as defined herein, encompasses the upper airways, including the oropharynx and larynx, followed by the lower airways, which include the trachea followed by bifurcations into the bronchi and bronchioli (e.g., terminal and respiratory). The upper and lower airways are called the conducting airways. The terminal bronchioli then divide into respiratory bronchioli which then lead to the ultimate respiratory zone, namely, the alveoli, or deep lung. The deep lung, or alveoli, are typically the desired target of inhaled therapeutic formulations for systemic drug delivery. “Pulmonary pH range,” as that term is used herein, refers to the pH range which can be encountered in the lung of a patient. Typically, in humans, this range of pH is from about 6.4 to about 7.0, such as from 6.4 to about 6.7. pH values of the airway lining fluid (ALF) have been reported in “Comparative Biology of the Normal Lung”, CRC Press, (1991) by R. A. Parent and range from 6.44 to 6.74.

Therapeutic, prophylactic or diagnostic agents, can also be referred to herein as “bioactive agents,” “medicaments” or “drugs.” The amount of therapeutic, prophylactic or diagnostic agent present in the particles can range from about 0.1 weight % to about 95 weight percent. In one embodiment, the amount of therapeutic, prophylactic or diagnostic agent present in the particles is 100 weight percent. In other embodiments, the amount of bioactive agent in the particles is approximately 10% to 50%, for example, 5%, 10%, 15%, 20%, 25%, 30%, 35%, 40%, 45%, 50% or 55%.

Combinations of bioactive agents also can be employed. Particles in which the drug is distributed throughout a particle are preferred. Suitable bioactive agents include agents which can act locally, systemically or a combination thereof. The term “bioactive agent,” as used herein, is an agent, or its pharmaceutically acceptable salt, which when released in vivo, possesses the desired biological activity, for example, therapeutic, diagnostic and/or prophylactic properties in vivo.

Examples of bioactive agent include, but are not limited to, synthetic inorganic and organic compounds, proteins and peptides, polysaccharides and other sugars, lipids, and DNA and RNA nucleic acid sequences having therapeutic, prophylactic or diagnostic activities. Agents with a wide range of molecular weight, for example, between 100 and 500,000 grams or more per mole can be used.

The agents can have a variety of biological activities, such as vasoactive agents, neuroactive agents, hormones, anticoagulants, immunomodulating agents, cytotoxic agents, prophylactic agents, antibiotics, antivirals, antisense, antigens, antineoplastic agents and antibodies.

Proteins include complete proteins, muteins and active fragments thereof, such as insulin, immunoglobulins, antibodies, cytokines (e.g., lymphokines, monokines, chemokines), interleukins, interferons (β-IFN, α-IFN and γ-IFN), erythropoietin, nucleases, tumor necrosis factor, colony stimulating factors, enzymes (e.g., superoxide dismutase, tissue plasminogen activator), tumor suppressors, blood proteins, hormones and hormone analogs (e.g., growth hormone, adrenocorticotropic hormone and luteinizing hormone releasing hormone (LHRH)), vaccines (e.g., tumoral, bacterial and viral antigens), antigens, blood coagulation factors; growth factors; granulocyte colony-stimulating factor (“G-CSF”); peptides include protein inhibitors, protein antagonists, protein agonists, calcitonin; nucleic acids include, for example, antisense molecules, oligonucleotides, and ribozymes. Polysaccharides, such as heparin, can also be administered. A particularly useful bioactive agent is insulin including, but not limited to, Humulin® Lente® (Humulin® L; human insulin zinc suspension), Humulin® R (regular soluble insulin (RI)), Humulin® Ultralente® (Humulin® U), and Humalog® 100 (insulin lispro (IL)) from Eli Lilly Co. (Indianapolis, Ind.; 100 U/mL).

Bioactive agents for local delivery within the lung, include agents such as those for the treatment of asthma, chronic obstructive pulmonary disease (COPD), emphysema, or cystic fibrosis. For example, genes for the treatment of diseases such as cystic fibrosis can be administered, as can beta agonists steroids, anticholinergics, and leukotriene modifers for asthma.

Other specific bioactive agents include, estrone sulfate, albuterol sulfate, parathyroid hormone-related peptide, somatostatin, nicotine, clonidine, salicylate, cromolyn sodium, salmeterol, formeterol, L-dopa, carbidopa or a combination thereof, gabapenatin, clorazepate, carbamazepine and diazepam.

Nucleic acid sequences include genes, antisense molecules which can, for instance, bind to complementary DNA to inhibit transcription, and ribozymes.

The particles can include any of a variety of diagnostic agents to locally or systemically deliver the agents following administration to a patient. For example, imaging agents which include commercially available agents used in positron emission tomography (PET), computer assisted tomography (CAT), single photon emission computerized tomography, x-ray, fluoroscopy, and magnetic resonance imaging (M1RI) can be employed.

Examples of suitable materials for use as contrast agents in MRI include the gadolinium chelates currently available, such as diethylene triamine pentacetic acid (DTPA) and gadopentotate dimeglumine, as well as iron, magnesium, manganese, copper and chromium.

Examples of materials useful for CAT and x-rays include iodine based materials for intravenous administration, such as ionic monomers typified by diatrizoate and iothalamate and ionic dimers, for example, ioxagalte.

Diagnostic agents can be detected using standard techniques available in the art and commercially available equipment.

The particles can further comprise a carboxylic acid which is distinct from the agent and lipid, in particular a phospholipid. In one embodiment, the carboxylic acid includes at least two carboxyl groups. Carboxylic acids, include the salts thereof as well as combinations of two or more carboxylic acids and/or salts thereof. In a preferred embodiment, the carboxylic acid is a hydrophilic carboxylic acid or salt thereof. Suitable carboxylic acids include but are not limited to hydroxydicarboxylic acids, hydroxytricarboxylic acids and the like. Citric acid and citrates, such as, for example, sodium citrate, are preferred. Combinations or mixtures of carboxylic acids and/or their salts also can be employed.

The carboxylic acid can be present in the particles in an amount ranging from about 0 weight % to about 80 weight %. Preferably, the carboxylic acid can be present in the particles in an amount of about 10% to about 20%, for example 5%, 10%, 15%, 20%, or 25%.

The particles suitable for use in the invention can further comprise an amino acid. In a preferred embodiment the amino acid is hydrophobic. Suitable naturally occurring hydrophobic amino acids, include but are not limited to, leucine, isoleucine, alanine, valine, phenylalanine, glycine and tryptophan. Combinations of hydrophobic amino acids can also be employed Non-naturally occurring amino acids include, for example, beta-amino acids. Both D, L configurations and racemic mixtures of hydrophobic amino acids can be employed. Suitable hydrophobic amino acids can also include amino acid derivatives or analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of unsaturation. Aromatic or aryl groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

A number of the suitable amino acids, amino acids analogs and salts thereof can be obtained commercially. Others can be synthesized by methods known in the art. Synthetic techniques are described, for example, in Green and Wuts, “Protecting Groups in Organic Synthesis, ”John Wiley and Sons, Chapters 5 and 7, 1991.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term hydrophobic amino acid refers to an amino acid that, on the hydrophobicity scale has a value greater or equal to 0.5, in other words, has a tendency to partition in the nonpolar acid which is at least equal to that of glycine.

Examples of amino acids which can be employed include, but are not limited to: glycine, proline, alanine, cysteine, methionine, valine, leucine, tyrosine, isoleucine, phenylalanine, tryptophan. Preferred hydrophobic amino acids include leucine, isoleucine, alanine, valine, phenylalanine, glycine and tryptophan. Combinations of hydrophobic amino acids can also be employed. Furthermore, combinations of hydrophobic and hydrophilic (preferentially partitioning in water) amino acids, where the overall combination is hydrophobic, can also be employed. Combinations of one or more amino acids can also be employed.

The amino acid can be present in the particles of the invention in an amount from about 0 weight % to about 60 weight %. Preferably, the amino acid can be present in the particles in an amount ranging from about 5 weight % to about 30 weight %. The salt of a hydrophobic amino acid can be present in the particles of the invention in an amount of from about 0 weight % to about 60 weight %. Preferably, the amino acid salt is present in the particles in an amount ranging from about 5 to about 30 weight %. Methods of forming and delivering particles which include an amino acid are described in U.S. Pat. application Ser. No. 09/382,959, filed on Aug. 25, 1999, entitled Use of Simple Amino Acids to Form Porous Particles During Spray Drying, and U.S. Pat. application Ser. No. 09/644,320, filed on Aug. 23, 2000, entitled Use of Simple Amino Acids to Form Porous Particles, the entire teachings of which are incorporated herein by reference.

In a further embodiment, the particles can also include other materials such as, for example, buffer salts, dextran, polysaccharides, lactose, trehalose, cyclodextrins, proteins, peptides, polypeptides, fatty acids, fatty acid esters, inorganic compounds, phosphates.

In one embodiment of the invention, the particles can further comprise polymers. The use of polymers can further prolong release. Biocompatible or biodegradable polymers are preferred. Such polymers are described, for example, in U.S. Pat. No. 5,874,064, issued on Feb. 23, 1999 to Edwards et al., the teachings of which are incorporated herein by reference in their entirety.

In yet another embodiment, the particles include a surfactant other than one of the charged lipids described above. As used herein, the term “surfactant” refers to any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent.

Suitable surfactants which can be employed in fabricating the particles of the invention include but are not limited to hexadecanol; fatty alcohols such as polyethylene glycol (PEG); polyoxyethylene-9-lauryl ether; a surface active fatty acid, such as palmitic acid or oleic acid; glycocholate; surfactin; a poloxomer; a sorbitan fatty acid ester such as sorbitan trioleate (Span 85); and tyloxapol.

The surfactant can be present in the particles in an amount ranging from about 0 weight % to about 60 weight %. Preferably, it can be present in the particles in an amount ranging from about 5 weight % to about 50 weight %.

It is understood that when the particles includes a carboxylic acid, a multivalent salt, an amino acid, a surfactant or any combination thereof that interaction between these components of the particle and the charged lipid can occur.

The particles, also referred to herein as powder, can be in the form of a dry powder suitable for inhalation. In a particular embodiment, the particles can have a tap density of less than about 0.4 g/cm³. Particles which have a tap density of less than about 0.4 g/cm³ (e.g., 0.4 g/cm³) are referred to herein as “aerodynamically light particles”. More preferred are particles having a tap density less than about 0.1 g/cm³ (e.g., 0.1 g/cm³).

Aerodynamically light particles have a preferred size, e.g., a volume median geometric diameter (VMGD) of at least about 5 microns (μm). In one embodiment, the VMGD is from about 5 μm to about 30 μm (for example, 5, 10, 15, 20, 25 or 30 μm). In another embodiment of the invention, the particles have a VMGD ranging from about 9 μm to about 30 μm. In other embodiments, the particles have a median diameter, mass median diameter (MMD), a mass median envelope diameter (MMED) or a mass median geometric diameter (MMGD) of at least 5 μm, for example, from about 5 μm to about 30 μm (for example, 5, 10, 15, 20, 25 or 30 μm), or from about 7 μm to about 8 μm (for example, 6 μm, 7 μm, or 8 μm).

Aerodynamically light particles preferably have “mass median aerodynamic diameter” (MMAD), also referred to herein as “aerodynamic diameteras”, between about 1 μm and about 5 μm (for example 1, 2, 3, 4, or 5 μm). In one embodiment of the invention, the MMAD is between about 1 μm and about 3 μm. In another embodiment, the MMAD is between about 3 μm and about 5 μm.

In another embodiment of the invention, the particles have an envelope mass density, also referred to herein as “mass density” of less than about 0.4 g/cm³. The envelope mass density of an isotropic particle is defined as the mass of the particle divided by the minimum sphere envelope volume within which it can be enclosed.

Tap density can be measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga. 30093). Tap density is a standard measure of the envelope mass density. Tap density can be determined using the method of USP Bulk Density and Tapped Density, United States Pharmacopia convention, Rockville, Md., 10^{th} Supplement, 4950-4951, 1999. Features which can contribute to low tap density include irregular surface texture and porous structure.

The diameter of the particles, for example, their VMGD, can be measured using an electrical zone sensing instrument such as a Multisizer IIe, (Coulter Electronic, Luton, Beds, England), or a laser diffraction instrument (for example, Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition within targeted sites within the respiratory tract.

Experimentally, aerodynamic diameter can be determined by employing a gravitational settling method, whereby the time for an ensemble of particles to settle a certain distance is used to infer directly the aerodynamic diameter of the particles. An indirect method for measuring the mass median aerodynamic diameter (MMAD) is the multi-stage liquid impinger (MSLI).

The aerodynamic diameter, dₐₑᵣ, can be calculated from the equation:

dₐₑᵣ=d_{g}√ρₜₐₚ

where d_{g} is the geometric diameter, for example, the MMGD and ρ is the powder density.

Particles which have a tap density less than about 0.4 g/cm³, median diameters of at least about 5 μm, and an aerodynamic diameter of between about 1 μm and about 5 μm, preferably between about 1 μm and about 3 μm, are more capable of escaping inertial and gravitational deposition in the oropharyngeal region, and are targeted to the airways or the deep lung. The use of larger, more porous particles is advantageous since they are able to aerosolize more efficiently than smaller, denser aerosol particles such as those currently used for inhalation therapies.

In comparison to smaller particles the larger aerodynamically light particles, preferably having a VMGD of at least about 5 μm, also can potentially more successfully avoid phagocytic engulfment by alveolar macrophages and clearance from the lungs, due to size exclusion of the particles from the phagocytes' cytosolic space. Phagocytosis of particles by alveolar macrophages diminishes precipitously as particle diameter increases beyond about 3 μm. Kawaguchi, H., et al., Biomaterials 7: 61-66 (1986); Krenis, L. J. and Strauss, B., Proc. Soc. Exp. Med., 107: 748-750 (1961); and Rudt, S. and Muller, R. H., J. Contr. Rel., 22: 263-272 (1992). For particles of statistically isotropic shape, such as spheres with rough surfaces, the particle envelope volume is approximately equivalent to the volume of cytosolic space required within a macrophage for complete particle phagocytosis.

The particles may be fabricated with the appropriate material, surface roughness, diameter and tap density for localized delivery to selected regions of the respiratory tract such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an aerodynamic diameter ranging from about 3 to about 5 μm are preferred for delivery to the central and upper airways. Particles having an aerodynamic diameter ranging from about 1 to about 3 μm are preferred for delivery to the deep lung.

In one embodiment, particles of the instant invention have an aerodynamic diameter of about 1.3 microns and a mean geometric diameter at 2 bar/16 mbar pressure of about 7.5 microns. In another embodiment, particles have about 44-45% of the particles with a fine particle fraction (FPF) less than about 3.4 microns, as detected using a 2 stage Anderson Cascade Impactor (ACI) assay. In another embodiment, particles have about 63-66% of the particles with a fine particle fraction of less than about 5.6 microns. Methods of measuring fine particle fraction using a 2 stage ACI assay are well known to those skilled in the art. One example of such an assay is as follows. Fine Particle Fractions (FPF) are measured using a reduced Thermo Anderson Cascade Impactor with two stages. Ten milligrams of powder are weighed into a size 2 hydroxpropyl methyl cellulose (HPMC) capsule. The powders are dispersed using a single-step, breath-actuated dry powder inhaler operated at 60 L/min for 2 seconds. The stages are selected to collect particles of an effective cutoff diameter (ECD) of (1) between 5.6 microns and 3.4 microns and (2) less than 3.4 microns and are fitted with porous filter material to collect the powder deposited. The mass deposited on each stage is determined gravimetrically. FPF is then expressed as a fraction of the total mass loaded into the capsule.

In another embodiment, particles of the instant invention have a mean geometric diameter at 1 bar of about 7 to about 8 microns as determined by RODOS. In another embodiment, particles have about 35% to about 40%, about 40% to about 45%, or about 45% to about 50% of the particles with a fine particle fraction of less than about 3.3 microns, as measured using a 3 stage ACI assay, as described herein.

Inertial impaction and gravitational settling of aerosols are predominant deposition mechanisms in the airways and acini of the lungs during normal breathing conditions. Edwards, D.A., J. Aerosol Sci., 26: 293-317 (1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1 μm), diminishing the tap density by increasing particle surface irregularities and particle porosity permits the delivery of larger particle envelope volumes into the lungs, all other physical parameters being equal.

The low tap density particles have a small aerodynamic diameter in comparison to the actual envelope sphere diameter. The aerodynamic diameter, dₐₑᵣ, is related to the envelope sphere diameter, d (Gonda, I., “Physico-chemical principles in aerosol delivery,” in Topics in Pharmaceutical Sciences 1991 (eds. D. J. A. Crommelin and K. K. Midha), pp. 95-117, Stuttgart: Medpharm Scientific Publishers, 1992)), by the formula:

dₐₑᵣ=d√ρ

where the envelope mass ρ is in units of g/cm³. Maximal deposition of monodispersed aerosol particles in the alveolar region of the human lung (˜60%) occurs for an aerodynamic diameter of approximately dₐₑᵣ=3 μm. Heyder, J. et al., J. Aerosol Sci., 17: 811-825 (1986). Due to their small envelope mass density, the actual diameter d of aerodynamically light particles comprising a monodisperse inhaled powder that will exhibit maximum deep-lung deposition is:

d=3/√ρ μm (where ρ<1 g/cm³);

where d is always greater than 3 μm. For example, aerodynamically light particles that display an envelope mass density, ρ=0.1 g/cm³, will exhibit a maximum deposition for particles having envelope diameters as large as 9.5 μm. The increased particle size diminishes interparticle adhesion forces. Visser, J., Powder Technology, 58: 1-10. Thus, large particle size increases efficiency of aerosolization to the deep lung for particles of low envelope mass density, in addition to contributing to lower phagocytic losses.

The aerodyanamic diameter can be calculated to provide for maximum deposition within the lungs, previously achieved by the use of very small particles of less than about five microns in diameter, preferably between about one and about three microns, which are then subject to phagocytosis. Selection of particles which have a larger diameter, but which are sufficiently light (hence the characterization “aerodynamically light”), results in an equivalent delivery to the lungs, but the larger size particles are not phagocytosed. Improved delivery can be obtained by using particles with a rough or uneven surface relative to those with a smooth surface.

Suitable particles can be fabricated or separated, for example, by filtration or centrifugation, to provide a particle sample with a preselected size distribution. For example, greater than about 30%, 50%, 70%, or 80% of the particles in a sample can have a diameter within a selected range of at least about 5 μm. The selected range within which a certain percentage of the particles must fall may be for example, between about 5 and about 30 μm, or optimally between about 5 and about 15 μm. In one preferred embodiment, at least a portion of the particles have a diameter between about 9 and about 11 μm. Optionally, the particle sample also can be fabricated wherein at least about 90%, or optionally about 95% or about 99%, have a diameter within the selected range. The presence of the higher proportion of the aerodynamically light, larger diameter particles in the particle sample enhances the delivery of therapeutic or diagnostic agents incorporated therein to the deep lung. Large diameter particles generally mean particles having a median geometric diameter of at least about 5 μm.

The particles can be prepared by spray drying. For example, a spray drying mixture, also referred to herein as “feed solution” or “feed mixture”, which includes the bioactive agent and one or more charged lipids having a charge opposite to that of the active agent upon association are fed to a spray dryer.

For example, when employing a protein active agent, the agent may be dissolved in a buffer system above or below the pI of the agent. Specifically, insulin, for example, may be dissolved in an aqueous buffer system (e.g., citrate, phosphate, acetate, etc.) or in 0.01 N HC1. The pH of the resultant solution then can be adjusted to a desired value using an appropriate base solution (e.g., 1 N NaOH). In one preferred embodiment, the pH may be adjusted to about pH 7.4. At this pH, insulin molecules have a net negative charge (pI=5.5). In another embodiment, the pH may be adjusted to about pH 4.0. At this pH, insulin molecules have a net positive charge (pI=5.5). In addition, if desired, the solutions can be heated to temperatures below their boiling points, for example, approximately 50° C. Typically the cationic phospholipid is dissolved in an organic solvent or combination of solvents. The two solutions are then mixed together and the resulting mixture is spray dried.

Suitable organic solvents that can be present in the mixture being spray dried include, but are not limited to, alcohols, for example, ethanol, methanol, propanol, isopropanol, butanols, and others. Other organic solvents include, but are not limited to, perfluorocarbons, dichloromethane, chloroform, ether, ethyl acetate, methyl tert-butyl ether and others. Aqueous solvents that can be present in the feed mixture include water and buffered solutions. Both organic and aqueous solvents can be present in the spray-drying mixture fed to the spray dryer. In one embodiment, an ethanol water solvent is preferred with the ethanol:water ratio ranging from about 50:50 to about 90:10. The mixture can have a neutral, acidic or alkaline pH. Optionally, a pH buffer can be included. Preferably, the pH can range from about 3 to about 10.

The total amount of solvent or solvents being employed in the mixture being spray dried generally is greater than about 98 weight percent. The amount of solids (drug, charged lipid and other ingredients) present in the mixture being spray dried can vary from about 1.0 weight percent to about 1.5 weight percent.

Using a mixture which includes an organic and an aqueous solvent in the spray drying process allows for the combination of hydrophilic and hydrophobic components, while not requiring the formation of liposomes or other structures or complexes to facilitate solubilization of the combination of such components within the particles.

Suitable spray-drying techniques are described, for example, by K. Masters in “Spray Drying Handbook,” John Wiley & Sons, New York, 1984. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a rotary atomizer is employed. An example of a suitable spray dryer using rotary atomization includes the Mobile Minor spray dryer, manufactured by Niro, Denmark. The hot gas can be, for example, air, nitrogen or argon.

Preferably, the particles of the invention are obtained by spray drying using an inlet temperature between about 100° C. and about 400° C. and an outlet temperature between about 50° C. and about 130° C.

The spray dried particles can be fabricated with a rough surface texture to reduce particle agglomeration and improve flowability of the powder. The spray-dried particle can be fabricated with features which enhance aerosolization via dry powder inhaler devices, and lead to lower deposition in the mouth, throat and inhaler device.

The particles of the invention can be employed in compositions suitable for drug delivery via the pulmonary system. For example, such compositions can include the particles and a pharmaceutically acceptable carrier for administration to a patient, preferably for administration via inhalation. The particles can be co-delivered with other similarly manufactured particles that may or may not contain yet another drug. Methods for co-delivery of particles is disclosed in U.S. Pat. application Ser. No. 09/878,146, filed Jun. 8, 2001, the entire teachings of which are incorporated herein by reference. The particles can also be co-delivered with larger carrier particles, not including a therapeutic agent, the latter possessing mass median diameters, for example, in the range between about 50 μm and about 100 μm. The particles can be administered alone or in any appropriate pharmaceutically acceptable carrier, such as a liquid, for example, saline, or a powder, for administration to the respiratory system.

Particles including a medicament, for example, one or more of drugs, are administered to the respiratory tract of a patient in need of treatment, prophylaxis or diagnosis. Administration of particles to the respiratory system can be by means such as those known in the art. For example, particles are delivered from an inhalation device. In a preferred embodiment, particles are administered via a dry powder inhaler (DPI). Metered-dose-inhalers (MDI), nebulizers or instillation techniques also can be employed.

Various suitable devices and methods of inhalation which can be used to administer particles to a patient's respiratory tract are known in the art. For example, suitable inhalers are described in U.S. Pat. No. 4,069,819, issued Aug. 5, 1976 to Valentini, et al., U.S. Pat. No.4,995,385 issued Feb. 26, 1991 to Valentini, et al., and U.S. Pat. No. 5,997,848 issued Dec. 7, 1999 to Patton, et al. Other examples include, but are not limited to, the Spinhaler® (Fisons, Loughborough, U.K.), Rotahaler® (Glaxo-Wellcome, Research Triangle Technology Park, N.C.), FlowCaps® (Hovione, Loures, Portugal), Inhalator® (Boehringer-Ingelheim, Germany), and the Aerolizer® (Novartis, Switzerland), the diskhaler (Glaxo-Wellcome, RTP, N.C.) and others, such as those known to those skilled in the art. Preferably, the particles are administered as a dry powder via a dry powder inhaler.

In one embodiment, the dry powder inhaler is a simple, breath actuated device. An example of a suitable inhaler which can be employed is described in U.S. patent application, entitled Inhalation Device and Method, by David A. Edwards, et al., filed on Apr. 16, 2001 under Attorney Docket No. 00166.0109.US00. The entire contents of this application are incorporated by reference herein. This pulmonary delivery system is particularly suitable because it enables efficient dry powder delivery of small molecules, proteins and peptide drug particles deep into the lung. Particularly suitable for delivery are the unique porous particles, such as the insulin particles described herein, which are formulated with a low mass density, relatively large geometric diameter and optimum aerodynamic characteristics (Edwards et al., 1998). These particles can be dispersed and inhaled efficiently with a simple inhaler device, as low forces of cohesion allow the particles to deaggregate easily. In particular, the unique properties of these particles confers the capability of being simultaneously dispersed and inhaled.

In one embodiment, the volume of the receptacle is at least about 0.37 cm³. In another embodiment, the volume of the receptacle is at least about 0.48 cm³. In yet another embodiment, are receptacles having a volume of at least about 0.67 cm³ or 0.95 cm³. The invention is also drawn to receptacles which are capsules, for example, capsules designated with a particular capsule size, such as 2, 1, 0, 00 or 000. Suitable capsules can be obtained, for example, from Shionogi (Rockville, Md.). Blisters can be obtained, for example, from Hueck Foils, (Wall, N.J.). Other receptacles and other volumes thereof suitable for use in the instant invention are known to those skilled in the art.

The receptacle encloses or stores particles and/or respirable compositions comprising particles. In one embodiment, the particles and/or respirable compositions comprising particles are in the form of a powder. The receptacle is filled with particles and/or compositions comprising particles, as known in the art. For example, vacuum filling or tamping technologies may be used. Generally, filling the receptacle with powder can be carried out by methods known in the art. In one embodiment of the invention, the particles which are enclosed or stored in a receptacle have a mass of at least about 5 milligrams. In another embodiment, the mass of the particles stored or enclosed in the receptacle comprises a mass of bioactive agent from at least about 1.5 mg to at least about 20 milligrams.

Preferably, particles administered to the respiratory tract travel through the upper airways (oropharynx and larynx), the lower airways, which include the trachea followed by bifurcations into the bronchi and bronchioli and through the terminal bronchioli which in turn divide into respiratory bronchioli leading then to the ultimate respiratory zone, the alveoli or the deep lung. In a preferred embodiment of the invention, most of the mass of particles deposits in the deep lung. In another embodiment of the invention, delivery is primarily to the central airways. Delivery to the upper airways can also be obtained.

In one embodiment of the invention, delivery to the pulmonary system of particles is in a single, breath-actuated step, as described in U.S. patent application entitled, “High Efficient Delivery of a Large Therapeutic Mass Aerosol,” application Ser. No. 09/591,307, filed Jun. 9, 2000, and continuation-in-part of U.S. patent application Ser. No. 09/878,146, entitled, “Highly Efficient Delivery of a Large Therapeutic Mass Aerosol,” filed Jun. 8, 2001, the entire teachings of which are incorporated herein by reference. In one embodiment, the dispersing and inhalation occurs simultaneously in a single inhalation in a breath-actuated device. An example of a suitable inhaler which can be employed is described in U.S. patent application, entitled “Inhalation Device and Method,” by David A. Edwards, et al., filed on Apr. 16, 2001 under Attorney Docket No. 00166.0109.US00. The entire contents of this application are incorporated by reference herein. In another embodiment of the invention, at least 50% of the mass of the particles stored in the inhaler receptacle is delivered to a subject's respiratory system in a single, breath-activated step.

In one further embodiment, at least 1.5 milligrams, or at least 5 milligrams, or at least 10 milligrams of a bioactive agent is delivered by administering, in a single breath, to a subject's respiratory tract particles enclosed in the receptacle. Amounts of bioactive agent as high as 15 milligrams can be delivered.

As used herein, the term “effective amount” means the amount needed to achieve the desired therapeutic or diagnostic effect or efficacy. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations (e.g., by means of an appropriate, conventional pharmacological protocol). In one embodiment, depending upon the patient, the dosage range is from about 40 IU to about 540 IU. Also, depending upon the patient, preferred dosage ranges are from about 84 IU to about 294 IU. Another effective dosage range for inhaled insulin is about 155 IU to about 170 IU. A useful conversion factor used herein is 27 IU for each 1 milligram of bioactive agent, in particular, insulin.

Aerosol dosage, formulations and delivery systems also may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles, Diagnosis and Therapy, Moren, et al., Eds, Esevier, Amsterdam, 1985.

As mentioned above, drug release rates can be described in terms of release constants. The first order release constant can be expressed using the following equations:

M₍ₜ₎=M₍ₒₒ₎*(1−e^{-k*t}) (1)

Where k is the first order release constant. M₍ₒₒ₎ is the total mass of drug in the drug delivery system, e.g. the dry powder, and M₍ₜ₎ is the amount of drug mass released from dry powders at time t.

Equations (1) may be expressed either in amount (i.e., mass) of drug released or concentration of drug released in a specified volume of release medium. For example, Equation (1) may be expressed as:

C₍ₜ₎=C₍ₒₒ₎*(1−e^{-k*t}) or Release ₍ₜ₎=Release₍ₒₒ₎*(1−e^{-k*t}) (2)

Where k is the first order release constant. C₍ₒₒ₎ is the maximum theoretical concentration of drug in the release medium, and C₍ₜ₎ is the concentration of drug being released from dry powders to the release medium at time t.

Drug release rates in terms of first order release constant can be calculated using the following equations:

k=−1n(M₍ₒₒ₎−M₍ₜ₎)/M₍ₒₒ₎/t (3)

The release constants presented in Table 5 employ equation (2).

As used herein, the term “a” or “an” refers to one or more.

The term “nominal dose” as used herein, refers to the total mass of bioactive agent which is present in the mass of particles targeted for administration and represents the maximum amount of bioactive agent available for administration.

Applicants' technology is based upon pulmonary delivery of dry powder aerosols composed of large, porous particles wherein each individual particle is capable of comprising both drug and excipient within a porous matrix. The particles are geometrically large but have low mass density and aerodynamic size. This results in a powder that is easily dispersible. The ease of dispersibility of the dry powder aerosols of large porous particles described herein allows for efficient systemic delivery of protein therapeutics from simple, breath activated, capsule based inhalers.

The invention also features a kit comprising at least two receptacles, each receptacle containing a different amount of dry powder insulin suitable for inhalation. The powder can be, but is not limited to any such dry powder insulin as described herein. In addition, the invention also features a kit comprising two or more receptacles comprising two or more unit dosages comprising particles comprising the bioactive agent formulations described herein. Depending on the bioavailability of the bioactive agent in the formulation, the formulation can contain more bioactive agent than the amount that is delivered to the subject's bloodstream. For example, as described in the Examples section below, a unit dosage of 42 IU, 84 IU, etc, can be contained in the receptacle administered to the subject, yet if the bioavailablility is less than 100%, then only a portion of the bioactive agent reaches the subject's bloodstream.

In one embodiment, the bioactive agent is insulin. For example, the formulation can be particles comprising, by weight, approximately 60% DPPC, approximately 30% insulin and approximately 10% sodium citrate; or comprising, by weight, approximately 40% DPPC, approximately 50% insulin and approximately 10% sodium citrate; or comprising by weight, approximately 40% to approximately 60% DPPC, approximately 30% to approximately 50% insulin and approximately 10% sodium citrate; or comprising by weight, approximately 80% DPPC, approximately 10% insulin and approximately 10% sodium citrate; or comprising, by weight, approximately 75% DPPC, approximately 15% insulin and approximately 10% sodium citrate; or comprising by weight, approximately 75% to approximately 80% DPPC, approximately 10% to approximately 15% insulin and approximately 10% sodium citrate. The formulation can be particles comprising, by weight, 60% DPPC, 30% insulin and 10% sodium citrate; or comprising, by weight, 40% DPPC, 50% insulin and 10% sodium citrate; or comprising by weight, 40% to 60% DPPC, 30% to 50% insulin and 10% sodium citrate; or comprising by weight, 80% DPPC, 10% insulin and 10% sodium citrate; or comprising, by weight, 75% DPPC, 15% insulin and 10% sodium citrate; or comprising by weight, 75% to 80% DPPC, 10% to 15% insulin and 10% sodium citrate. The desired dose can be achieved in a number of different ways. For example, the size of the receptacle can be varied and/or the volume of formulation loaded into the receptacle and/or the formulation (e.g., percent of insulin) can be varied in order to achieve the desired dose. The desired dose can be the dose in the receptacle, or the dose that is bioavailable to the subject (e.g., the amount released into the subject's bloodstream). When the receptacle is only partially filled with the formulation, the remainder of the receptacle can remain empty or be loaded to 100% capacity with a filler.

The kits described herein can be used to deliver bioactive agents, for example, insulin to a subject in need of the bioactive agent. When the bioactive agent is insulin, the dose administered to the subject can be altered, for example, by a patient or by a medical provider, by increasing or decreasing the number of receptacles (e.g., capsules) of insulin containing particles, thereby increasing or decreasing the unit dosage of the insulin. When a patient is in need of a higher dose of insulin than usual, that patient can administer to himself or herself additional receptacles, or a different combination of receptacles, so that the dose of insulin is increased to the desired amount. Conversely, when a patient needs less insulin, the patient can administer to himself or herself fewer receptacles, or a different combination of receptacles, such that the dose is decreased to the desired amount. The kits may also contain instructions for the use of the reagents in the kits (e.g., the receptacles containing the formulation). Through the use of such kits, accurate dosing can be accomplished.

### EXEMPLIFICATION

### Materials

For the in vivo rat studies, bulk insulin for using spray drying was obtained from BioBras (Belo Horizonte, Brazil) or Sigma (Saint Loius, Mo.). For in vitro and human in vivo studies, Humulin® Lente® (Humulin® L human insulin zinc suspension), Humulin® R (regular soluble insulin (IR)), Humulin® Ultralente® (Humulin® U), and Humalog® 100 ( insulin lispro (IL)) were obtained from Eli Lilly Co. (Indianapolis, Ind.; 100 U/mL). These solutions were stored at 2-8° C.

### Mass Median Aerodynamic Diameter-MMAD (μm)

The mass median aerodynamic diameter was determined using an Aerosizer/Aerodisperser (Amherst Process Instrument, Amherst, Mass.). Approximately 2 mg of powder formulation was introduced into the Aerodisperser and the aerodynamic size was determined by time of flight measurements.

### Fine Particle Fraction

Fine particle fraction can be used as one way to characterize the aerosol performance of a dispersed powder. Fine particle fraction describes the size distribution of airborne particles. Gravimetric analysis, using Cascade impactors, is one method of measuring the size distribution, or fine particle fraction, of airborne particles. The Andersen Cascade Impactor (ACI) is an eight-stage impactor that can separate aerosols into nine distinct fractions based on aerodynamic size. The size cutoffs of each stage are dependent upon the flow rate at which the ACI is operated.

A 2 stage collapsed ACI can be used to measure fine particle fraction. The 2 stage collapsed ACI consists of only the top two stages of the eight-stage ACI and allows for the collection of two separate powder fractions. The ACI is made up of multiple stages consisting of a series of nozzles and an impaction surface. At each stage an aerosol stream passes through the nozzles and impinges upon the surface. Particles in the aerosol stream with a large enough inertia will impact upon the plate. Smaller particles that do not have enough inertia to impact on the plate will remain in the aerosol stream and be carried to the next stage. Each successive stage of the ACI has a higher aerosol velocity in the nozzles so that smaller particles can be collected at each successive stage.

The particles of the invention can be characterized by fine particle fraction. A 2 stage collapsed Andersen Cascade Impactor is used to determine fine particle fraction. Specifically, a two-stage collapsed ACI is calibrated so that the fraction of powder that is collected on stage one is composed of particles that have an aerodynamic diameter of less than 5.6 microns and greater than 3.4 microns. The fraction of powder passing stage one and depositing on a collection filter is thus composed of particles having an aerodynamic diameter of less than 3.4 microns. The airflow at such a calibration is approximately 60 L/min.

A 3 stage ACI can also be used to determine the fine particle fraction. The 3 stage ACI assay was carried out as follows. A 3-stage Andersen Cascade Impactor (ACI) (Andersen Instruments, Inc., Smyrna, Ga.) with screens was assembled and used to determine fine particle fraction. ACI stages 0, 2 and 3 with effective cutoff diameters of 9.0, 4.7, and 3.3 microns (at a flow rate of 28.3±2 L/min) were used in the apparatus. Each stage comprised an impaction plate, a screen, and a jet plate. The screens used were stainless steel 150 micron pore, 5-layer sintered Dynapore laminate (Martin Kurz & Co, Inc., Mineola, N.Y.). Screens were rinsed with methanol, allowed to dry, and then immersed in HPLC grade water and immediately placed on the solid impaction plates of the instrument. A pre-weighted 81 mm glass fiber filter (Anderson Instruments, Inc., Symyma, Ga.) was used as the instrument's filter medium.

Three-stage Andersen Cascade Impactor assays were conducted at 18 to 25° C. and 20 to 40% relative humidity. The air flow rate through the instrument was calibrated to 28.3±2 L/min. A capsule was filled with powder and placed inside an inhaler device. The capsule was then punctured using the inhaler and placed in a mouthpiece adaptor on the ACI. An air pump was activated for about 4.2 seconds to draw the powder from the capsule. The ACI was dissembled and the glass fiber filter was weighed. Fine particle fraction (FPF), less than 3.3 microns, was determined by dividing the mass of powder deposited on the filter by the total mass of powder loaded into the capsule.

The terms “FPF<5.6” and “fine particle fraction less than 5.6 microns,” as used herein, refer to the fraction of a sample of particles that have an aerodynamic diameter of less than 5.6 microns. FPF(<5.6) can be determined by dividing the mass of particles deposited on the stage one and on the collection filter of a 2 stage collapsed ACI by the mass of particles weighed into a capsule for delivery to the instrument.

The terms “FPF (<3.4)” and “fine particle fraction, less than 3.4 microns,” as used herein, refer to the fraction of a mass of particles that have an aerodynamic diameter of less than 3.4 microns. FPF(<3.4) can be determined by dividing the mass of particles deposited on the collection filter of a 2 stage collapsed ACI by the mass of particles weighed into a capsule for delivery to the instrument.

The terms “FPF (<3.3)” and “fine particle fraction less than 3.3 microns,” as used herein, refer to the fraction of a mass of particles that have an aerodynamic diameter of less than 3.4 microns. FPF(<3.3) can be determined by dividing the mass of particles deposited on the collection filter of a 3 stage collapsed ACI by the mass of particles weighed into a capsule for delivery to the instrument.

The “FPF less than 5.6” has been demonstrated to correlate to the fraction of the powder that is able to make it into the lung of the patient, while the “FPF less than 3.4” (using the 2 stage ACI) or “FPF less than 3.3” (using the 3 stage ACI) has been demonstrated to correlate to the fraction of the powder that reaches the deep lung of a patient. These correlations provide a quantitative indicator that can be used for particle optimization.

### Volume Median Geometric Diameter-VMGD (μm)

The volume median geometric diameter was measured using a RODOS dry powder disperser (Sympatec, Princeton, N.J.) in conjunction with a HELOS laser diffractometer (Sympatec). Powder was introduced into the RODOS inlet and aerosolized by shear forces generated by a compressed air stream regulated at 2 bar. The aerosol cloud was subsequently drawn into the measuring zone of the HELOS, where it scattered light from a laser beam and produced a Fraunhofer diffraction pattern used to infer the particle size distribution and determine the median value.

Where noted, the volume median geometric diameter was determined using a Coulter Multisizer II. Approximately 5-10 mg powder formulation was added to 50 mL isoton II solution until the coincidence of particles was between 5% and 8%.

### Determination of Plasma Insulin Levels in Rats

Quantification of insulin in rat plasma was performed using a human insulin specific RIA kit (Linco Research, Inc., St. Charles, Mo., catalog #HI-14K). The assay shows less than 0.1 % cross reactivity with rat insulin. The assay kit procedure was modified to accommodate the low plasma volumes obtained from rats, and had a sensitivity of approximately 5 μU/mL.

### Preparation of Insulin Formulations

The powder formulations listed in Table 2 were prepared as follows. Pre-spray drying solutions were prepared by dissolving the lipid in ethanol and the insulin, leucine, and/or sodium citrate in water. The ethanol solution was then mixed with the water solution at a ratio of 60/40 ethanol/water. Final total solute concentration of the solution used for spray drying varied from 1 g/L to 3 g/L. As an example, the DPPC/citrate/insulin (60/10/30) spray drying solution was prepared by dissolving 600 mg DPPC in 600 mL of ethanol, dissolving 100 mg of sodium citrate and 300 mg of insulin in 400 mL of water and then mixing the two solutions to yield one liter of cosolvent with a total solute concentration of 1 g/L (w/v). Higher solute concentrations of 3 g/L (w/v) were prepared by dissolving three times more of each solute in the same volumes of ethanol and water.

The solution was then used to produce dry powders. A Niro Atomizer Portable Spray Dryer (Niro, Inc., Columbus, Md.) was used. Compressed air with variable pressure (1 to 5 bar) ran a rotary atomizer (2,000 to 30,000 rpm) located above the dryer. Liquid feed with varying rate (20 to 66 mL/min) was pumped continuously by an electronic metering pump (LMI, Model #A151-192s) to the atomizer. Both the inlet and outlet temperatures were measured. The inlet temperature was controlled manually; it could be varied between 100° C. and 400° C. and was established at 100, 110, 150, 175 or 200° C., with a limit of control of 5° C. The outlet temperature was determined by the inlet temperature and such factors as the gas and liquid feed rates (it varied between 50° C. and 130° C.). A container was tightly attached to the cyclone for collecting the powder product.

TABLE 2
Insulin Powder Formulations

| POWDER | | | | | | | |
|---|---|---|---|---|---|---|---|
| FORMU- | COMPOSITION (%) | | | | | | |
| LATION | | | | | Leu- | | Insu- |
| NUMBER | DPePC | DSePC | DPPG | DPPC | cine | Citrate | lin |
| 1 | | | | 70 | 10 | | 20 |
| 2 | | 70 | | | 20 | | 10 |
| 3 | | 70 | | | 10 | | 20 |
| 4 | 50 | | | | | | 50 |
| 5 | | | 40 | | | 10 | 50 |
| 6 | 70 | | | | 10 | | 20 |
| 7 | 50 | | | | | | 50 |
| 8 | 54.5 | | | | | | 45.5 |
| 9 | 50 | | | | 10 | | 40 |
| 10 | 70 | | | | 10 | | 2 |
| 11 | 70 | | | | 8 | 2 | 20 |
| 12 | | | | 40 | | 10 | 50 |
| 13† | | | | 60 | | 10 | 30 |
| 13A† | | | | 60 | | 10 | 30 |

†Different lots of the same formulation.

The physical characteristic of the insulin containing powders is set forth in Table 3. The MMAD and VMGD were determined as detailed above.

TABLE 3
Physical Characteristics of Insulin Powder Formulations

| Formu- | Compositions | MMAD | VMGD | Density |
|---|---|---|---|---|
| lations | (% weight basis) | (μm) § | (μm) ¶ | (g/cc) ‡ |
| 1 | DPPC/Leu/Insulin (Sigma) = | 2.6 | 13.4 | 0.038 |
| | 70/10/20 | | | |
| 2 | DSePC (Avanti)/Leu/Insulin | 3.3 | 10.0 | 0.109 |
| | (Sigma) = 70/10/20 | | | |
| 3 | DSePC (Avanti)/Leu/Insulin | 3.4 | 13.6 | 0.063 |
| | (Sigma) = 70/10/20 | | | |
| 4 | DPePC (Avanti)/lnsuiln (Sigma) = | 3.2 | 15.3 | 0.044 |
| | 50/50 | | | |
| 5 | DPPG/Sodium Citrate/Insulin = | 3.9 | 11.6 | 0.113 |
| | 40/10/50 | | | |
| 6 | DPePC (Genzyme)/Leu/Insulin | 2.6 | 9.1 | 0.082 |
| | (BioBras) = 70/10/20 | | | |
| 7 | DPePC (Avanti)/Insulin | 2.8 | 11.4 | 0.060 |
| | (BioBras) = 50/50 | | | |
| 8 | DPePC (Genzyme)/Insulin | 2.8 | 12.6 | 0.049 |
| | (BioBras) = 54.5/45.5 | | | |
| 9 | DPePC (Genzyme)/Leu/Insulin | 2.2 | 8.4 | 0.069 |
| | (BioBras) = 50/10/40 | | | |
| 10 | DPePC (Avanti)/Leu/Insulin | 3.7 | 15.5 | 0.057 |
| | (BioBras) = 70/10/20 | | | |
| 11 | DPePC (Avanti)/Leu/Sodium | 2.6 | 15.3 | 0.029 |
| | Citrate/Insulin (BioBras) = | | | |
| | 70/8/2/20 | | | |
| 12 | DPPC/Sodium Citrate/Insulin = | 3.5 | 11.6 | 0.091 |
| | 40/10/50 | | | |
| 13 | DPPC/Insulin/Sodium Citrate = | 1.9 | 8.0 | 0.056 |
| | 60/30/10 | | | |

§ Mass median aerodynamic diameter
¶ Volumetric median geometric diameter at 2 bar pressure
‡ Determined using dₐₑᵣ = d_{g}√ρ

The data presented in Table 3 showing the physical characteristics of the formulations comprising insulin are predictive of the respirability of the formulations. That is, as discussed above, the large geometric diameters, small aerodynamic diameters and low densities possessed by the powder prepared as described herein render the particles highly respirable.

### Alternative Method for Preparation and Packagaing of b 30 Weight Percent Insulin Containing Particles

The following example describes the preparation of particles with a 30 wt % insulin load (DPPC/insulin/citrate, 60/30/10 wt %). The following procedure details preparation of a one liter solution batch. Batch preparation can be scaled accordingly to generate larger volumes of feed solution. Typical spray drying batch sizes for the Size 1 Niro spray dryer (see below) are approximately 24 liters. An aqueous solution was prepared as follows. 0.4 L of a pH 2.5 citrate buffer was prepared by dissolving 1.26 grams of citric acid monohydrate in 0.4 L of sterile water for injection and adjusting the pH to 2.5 with 1.0 N HCI. 4.5 grams of insulin were then dissolved into this citrate buffer. Finally, 1.0 N sodium hydroxide (NaOH) was added until the pH had been adjusted to 6.7. An organic solution was prepared by dissolving 9.0 g DPPC in 600 ML of ethanol (200 proof, USP).

Prior to spray drying, both the aqueous and organic solutions were in-line filtered (0.22 micron filter) and then in-line heated to 50° C. A spray-drying feed solution was prepared by in-line static mixing the heated aqueous solution with the heated organic solution. The resulting aqueous/organic feed solution was combined such that it had a final volumetric composition of 60% ethanol/40% water with a solute concentration of 15 grams/L. This feed solution was pumped at a controlled rate of 50 mL/min into the top of the spray-drying chamber (Size 1 Niro spray dryer, Model Mobil Minor 2000). Upon entering the spray-drying chamber, the solution was atomized into small droplets of liquid using a 2 fluid atomizer (Liquid Cap 2850 and Gas Cap 67147, Spraying Systems Inc) with an atomization gas rate of 70 g/min. The process gas, heated nitrogen maintained at −20° C. dew point, was introduced at a controlled rate of 94 kg/hr into the top of the drying chamber. As the liquid droplets contacted the heated nitrogen, the liquid evaporated and porous particles were formed. The temperature of the inlet drying gas was 135° C. and the outlet process gas temperature was 67.5° C. The particles exited the drying chamber with the process gas and entered a product filter downstream. The product filter separated the porous particles from the process gas stream. The process gas exited from the top of the collector and was directed to the exhaust system. Periodically, the filter was reverse pulsed and product exited from the bottom of the product filter and were recovered in a powder collection vessel.

Resulting particles had a tap density of 0.09g/cm³, determined using standard methods, a VMGD of 7 to 8 microns at 1 bar as determined by RODOS and a fine particle fraction (FPF)<3.3 microns of 45 to 50% as determined using a 3 stage ACI assay with wet screens, as described herein.

Powder was filled at approximately 8.7-mg quantities into size 2 hydroxypropylmethyl cellulose (HPMC) capsules and then packaged in Aclar-foil blister cards. The blister cards were sealed in aluminum foil bags, containing a small, food-grade desiccant bag for additional moisture protection.

### Alternative Method for Preparation and Packaging of 10 Weight Percent Insulin Containing Particles

The following section describes the preparation of particles with a 10 wt % insulin load (DPPC/insulin/citrate, 80/10/10 wt %). The following procedure details preparation of a one liter solution batch. An aqueous solution was prepared as follows. 0.4 L of a pH 2.5 citrate buffer was prepared by dissolving 0.168 grams of citric acid monohydrate in 0.4 L of sterile water for injection and adjusting the pH to 2.5 with 1.0N HCI. 0.2 grams of insulin were then dissolved into this citrate buffer. Finally, 1.0 N sodium hydroxide (NaOH) was added until the pH had been adjusted to 6.7. An organic solution was prepared by dissolving 1.2 g DPPC in 600 mL of ethanol (200 proof, USP).

Prior to spray drying, both the aqueous and organic solutions were in-line filtered (0.22 micron filter) and then in-line heated to 50° C. A spray-drying feed solution was prepared by in-line static mixing the heated aqueous solution with the heated organic solution. The resulting aqueous/organic feed solution was combined such that it had a final volumetric composition of 60% ethanol/40% water with a solute concentration of 2 grams/L. This feed solution was pumped at a controlled rate of 45 mL/min into the top of the spray-drying chamber (Size 1 Niro spray dryer, Model Mobil Minor 2000). Upon entering the spray-drying chamber, the solution was atomized into small droplets of liquid using a 2 fluid atomizer (Liquid Cap 2850 and Gas Cap 67147, Spraying Systems Inc) with an atomization gas rate of 21.5 g/min. The process gas, heated dry nitrogen, was introduced at a controlled rate of 90 kg/hr into the top of the drying chamber. As the liquid droplets contacted the heated nitrogen, the liquid evaporated and porous particles were formed. The temperature of the inlet drying gas was 130° C. and the outlet process gas temperature was 67.5° C. The particles exited the drying chamber with the process gas and entered a product filter downstream. The product filter separated the porous particles from the process gas stream. The process gas exited from the top of the collector and was directed to the exhaust system. Periodically, the filter was reverse pulsed and product exits from the bottom of the product filter and was recovered in a powder collection vessel.

Resulting particles had a tap density of 0.06g/cm³, determined using standard methods, a VMGD of 7 to 8 microns at 1 bar as determined by RODOS and an FPF<3.3 of 35 to 40% as determined using a 3 stage ACI assay with wet screens, as described herein. Powder was filled at approximately 12.4-mg quantities into size 2 hydroxypropylmethyl cellulose (HPMC) capsules and then packaged in Aclar-foil blister cards. The blister cards were sealed in aluminum foil bags, containing a small, food-grade desiccant bag for additional moisture protection.

### Method for Preparation and Packagaing of 15 Weight Percent Insulin Containing Particles

The following example describes the preparation of particles with a 15 wt% insulin load (DPPC/insulin/citrate, 75/15/10 wt%). The following procedure details preparation of a one liter solution batch. An aqueous solution was prepared as follows. 0.4 L of a pH 2.5 citrate buffer was prepared by dissolving 1.26 gr of citric acid monohydrate in 0.4 L of sterile water for injection and adjusting the pH to 2.5 with 1.0N HCI. 2.25 gr of insulin were then dissolved into this citrate buffer. Finally, 1.0 N sodium hydroxide (NaOH) was added until the pH had been adjusted to 6.7. An organic solution was prepared by dissolving 11.25 g DPPC in 600 mL of ethanol (200 proof, USP).

Prior to spray drying, both the aqueous and organic solutions were in-line filtered (0.22 micron filter) and then in-line heated to 50° C. A spray-drying feed solution was prepared by in-line static mixing the heated aqueous solution with the heated organic solution. The resulting aqueous/organic feed solution was combined such that it had a final volumetric composition of 60% ethanol/ 40% water with a solute concentration of 15 gr/L. This feed solution was pumped at a controlled rate of 50 mL/min into the top of the spray-drying chamber (Size 1 Niro spray dryer, Model Mobil Minor 2000). Upon entering the spray-drying chamber, the solution was atomized into small droplets of liquid using a 2 fluid atomizer (Liquid Cap 2850 and Gas Cap 67147, Spraying Systems Inc) with an atomization gas rate of 62 g/min. The process gas, heated dry nitrogen, was introduced at a controlled rate of 110 kg/hr into the top of the drying chamber. As the liquid droplets contacted the heated nitrogen, the liquid evaporated and porous particles were formed. The temperature of the inlet drying gas was 128° C. and the outlet process gas temperature was 67.5° C. The particles exited the drying chamber with the process gas and entered a product filter downstream. The product filter separated the porous particles from the process gas stream. The process gas exited from the top of the collector and was directed to the exhaust system. Periodically, the filter was reverse pulsed and product exited from the bottom of the product filter and was recovered in a powder collection vessel. Resulting particles had a VMGD of 7 to 8 microns at 1 bar as determined by RODOS and an FPF<3.3 of 40 to 45% as determined using a 3 stage ACI with wet screens. Powder was filled at approximately 8.0-mg quantities into size 2 hydroxypropylmethyl cellulose (HPMC) capsules and then packaged in Aclar-foil blister cards. The blister cards were sealed in aluminum foil bags, containing a small, food-grade desiccant bag for additional moisture protection.

### In Vivo Rat Insulin Experiments

The following experiment was performed to determine the rate and extent of insulin absorption into the blood stream of rats following pulmonary administration of dry powder formulations comprising insulin to rats.

The nominal insulin dose administered was 100 μg per rat. To achieve the nominal doses, the total weight of powder administered per rat ranged from 0.2 mg to 1 mg, depending on the composition of each powder. Male Sprague-Dawley rats were obtained from Taconic Farms (Germantown, N.Y.). At the time of use, the animals weighed 386 g in average (±5 g S.E.M.). The animals were allowed free access to food and water.

The powders were delivered to the lungs using an insufflator device for rats (PennCentury, Philadelphia, Pa.). The powder amount was transferred into the insufflator sample chamber. The delivery tube of the insufflator was then inserted through the mouth into the trachea and advanced until the tip of the tube was about a centimeter from the carina (first bifurcation). The volume of air used to deliver the powder from the insufflator sample chamber was 3 mL, delivered from a 10 mL syringe. In order to maximize powder delivery to the rat, the syringe was recharged and discharged two more times for a total of three air discharges per powder dose.

The injectable insulin formulation Humulin L was administered via subcutaneous injection, with an injection volume of 7.2 μL for a nominal dose of 25 μg insulin. Catheters were placed into the jugular veins of the rats the day prior to dosing. At sampling times, blood samples were drawn from the jugular vein catheters and immediately transferred to EDTA coated tubes. Sampling times were 0, 0.25, 0.5, 1, 2, 4, 6, 8, and 24 hrs. after powder administration. In some cases an additional sampling time (12 hrs.) was included, and/or the 24 hr. time point omitted. After centrifugation, plasma was collected from the blood samples. Plasma samples were stored at 4° C. if analysis was performed within 24 hours or at −75° C. if analysis would occur later than 24 hours after collection. The plasma insulin concentration was determined as described above.

Table 4 contains the insulin plasma levels quantified using the assay described above.

TABLE 4
Rat Insulin Plasma Levels

| Time | PLASMA INSULIN CONCENTRATION (μU/mL) ± S.E.M. | | | | | | | |
|---|---|---|---|---|---|---|---|---|
| (hrs) | Formu- | Formu- | Formu- | Formu- | Formu- | Formu- | Formu- | |
| ↓ | lation 1 | lation 2 | lation 3 | lation 4 | lation 5 | lation 6 | lation 13A | Humlin L |
| 0 | 5.0 ± | 5.2 ± | 5.0 ± | 5.0 ± | 5.3 ± | 5.7 ± | 5.0 ± | 5.0 ± |
| | 0.0 | 0.2 | 0.0 | 0.0 | 0.2 | 0.7 | 0.0 | 0.0 |
| 0.25 | 1256.4 ± | 61.6 ± | 98.5 ± | 518.2 ± | 240.8 ± | 206.8 ± | 1097.7 ± | 269.1 ± |
| | 144.3 | 22.5 | 25.3 | 179.2 | 67.6 | 35.1 | 247.5 | 82.8 |
| 0.5 | 1335.8 ± | 85.2 ± | 136.7 ± | 516.8 ± | 326.2 ± | 177.3 ± | 893.5 ± | 459.9 ± |
| | 81.9 | 21.7 | 37.6 | 190.9 | 166.9 | 7.8 | 177.0 | 91.6 |
| 1 | 859.0 ± | 85.4 ± | 173.0 ± | 497.0 ± | 157.3 ± | 170.5 ± | 582.5 ± | 764.7 ± |
| | 199.4 | 17.6 | 28.8 | 93.9 | 52.5 | 32.9 | 286.3 | 178.8 |
| 2 | 648.6 ± | 94.8 ± | 158.3 ± | 496.5 ± | 167.7 ± | 182.2 ± | 208.5 ± | 204.4 ± |
| | 171.1 | 25.0 | 39.1 | 104.9 | 70.5 | 75.0 | 78.3 | 36.7 |
| 4 | 277.6 ± | 52.5 ± | 98.0 ± | 343.8 ± | 144.8 ± | 170.2 ± | 34.9 ± | 32.1 ± |
| | 86.8 | 9.1 | 24.3 | 66.7 | 43.8 | 56.3 | 5.4 | 22.6 |
| 6 | 104.0 ± | 33.0 ± | 58.7 ± | 251.2 ± | 95.7 ± | 159.5 ± | 12.3 ± | 11.1 ± |
| | 43.1 | 10.7 | 4.1 | 68.4 | 27.3 | 43.4 | 2.4 | 7.5 |
| 8 | 54.4 ± | 30.2 ± | 42.5 ± | 63.2 ± | 52.5 ± | 94.8 ± | 5.2 ± | 5.5 ± |
| | 34.7 | 8.1 | 17.8 | 16.5 | 13.7 | 23.5 | 0.1 | 2.1 |
| 12 | | | | 17.2 ± | | | | |
| | | | | 6.5 | | | | |
| 24 | | | | 5.0 ± | 5.5 ± | | | |
| | | | | 0.0 | 0.3 | | | |

The in vivo release data of Table 4 show that powder formulations comprising insulin and the lipid DPPC (Formulations 1 and 13) have a more rapid release than, for example, powder formulations comprising insulin and positively charged lipids (DPePC and DSePC) which have sustained elevated levels at 6 to 8 hours.

### In Vitro Analysis of Insulin-Containing Formulations

The in vitro release of insulin containing dry powder formulations was performed as described by Gietz et al. in Eur. J. Pharm. Biopharm., 45:259-264 (1998), with several modifications. Briefly, in 20 mL screw-capped glass scintillation vials about 10 mg of each dry powder formulation or solution of Humulin R, Humulin L, or Humulin U was mixed with 4 mL of warm (37° C.) 1% agarose solution using polystyrene stir bars. The resulting mixture was then distributed in 1 mL aliquots to a set of five fresh 20 mL glass scintillation vials. The dispersion of dry powder in agarose was cooled in an ambient temperature dessicator box protected from light to allow gelling. Release studies were conducted on an orbital shaker at about 37° C. At predetermined time points, previous release medium (1.5 mL) was removed and fresh release medium (1.5 mL) was added to each vial. Typical time points for these studies were 5 minutes, and 1, 2, 4, 6 and 24 hours. The release medium used consisted of 20 mM 4-(2-hydroxyethyl)-piperazine-1-ethanesulfonic acid (HEPES), 138 mM NaCl, 0.5% Pluronic (Synperonic PE/F68; to prevent insulin filbrillation in the release medium); pH 7.4. A Pierce (Rockford, Ill.) protein assay kit (See Anal. Biochem., 150:76-85 (1985)) using known concentrations of insulin standard was used to monitor insulin concentrations in the release medium.

Table 5 summarizes the in vitro release data and first order release constants for powder formulations of Table 2 comprising insulin.

TABLE 5
In Vitro Insulin Release

| | | | Maximum ‡ | |
|---|---|---|---|---|
| | Cumulative | Cumulative | Release | |
| Formu- | % Insulin | % Insulin | at 24 hr | First Order ‡ |
| lation | Released | Released | (Cumulative | Release |
| Number | at 6 hr | at 24 hr | %) | Constants (hr⁻¹) |
| Humulin | 92.67 ± 0.36 | 94.88 ± 0.22 | 91.6 ± 5.42 | 1.0105 ± 0.2602 |
| R | | | | |
| (solution) | | | | |
| Humulin | 19.43 ± 0.41 | 29.71 ± 0.28 | 36.7 ± 2.56 | 0.0924 ± 0.0183 |
| L | | | | |
| (solution) | | | | |
| Humulin | 5.17 ± 0.18 | 12.65 ± 0.43 | 46.6 ± 27.0 | 0.0158 ± 0.0127 |
| U | | | | |
| (solution) | | | | |
| 2 | 31.50 ± 0.33 | 47.52 ± 0.43 | 48.22 ± 0.46 | 0.1749 ± 0.0038 |
| 3 | 26.34 ± 0.71 | 37.49 0.27 | 38.08 ± 0.72 | 0.1837 ± 0.0079 |
| 4 | 24.66 ± 0.20 | 31.58 ± 0.33 | 31.51 ± 1.14 | 0.2457 ± 0.0214 |
| 5 | 29.75 ± 0.17 | 35.28 ± 0.19 | 33.66 ± 2.48 | 0.4130 ± 0.0878 |
| 6 | 17.04 ± 0.71 | 24.71 ± 0.81 | 25.19 ± 0.52 | 0.1767 ± 0.0083 |
| 7 | 13.53 ± 0.19 | 19.12 ± 0.40 | 19.51 ± 0.48 | 0.1788 ± 0.0101 |
| 8 | 13.97 ± 0.27 | 17.81 ± 0.46 | 17.84 ± 0.55 | 0.2419 ± 0.0178 |
| 9 | 17.47 ± 0.38 | 22.17 ± 0.22 | 21.97 ± 0.64 | 0.2734 ± 0.0196 |
| 10 | 25.96 ± 0.31 | 34.94 ± 0.31 | 35.43 ± 0.90 | 0.2051 ± 0.0120 |
| 11 | 34.33 ± 0.51 | 47.21 ± 0.47 | 47.81 ± 0.85 | 0.1994 ± 0.0082 |
| 12 | 61.78 ± 0.33 | 68.56 ± 0.23 | 65.20 ± 3.34 | 0.5759 ± 0.0988 |
| 13 | 78.47 ± 0.40 | 85.75 ± 0.63 | 84.9 ± 3.81 | 0.5232 ± 0.0861 |

‡ Release₍ₜ₎ = Release_{(inf)} * (1 - e^{-k*t})
† Used as a control formulation.

### Human Clinical Trial

Described below is a human study of the clinical pharmacodynamic (PD) properties, safety and tolerability of a novel inhaled insulin engineered with unique aerodynamic properties. The euglycaemic clamp was used for assessing the metabolic activity of the insulin delivered to the subjects in the study by the inhaler. The clamp is a well described technique that allows the administration of insulin to normal volunteers or diabetic patients without the risk of hypoglycaemia (Heinemann et al., Metab. Res., 26:579-583 (1994); and Clemens et al., Clin. Chem., 28:1899-1904 (1982).

A dry powder formulation of inhaled insulin (60% DPPC, 30% insulin and 10% citrate) was compared with a fast acting commercial subcutaneous (s.c.) preparation of insulin lispro, as well as a fast acting s.c. formulation of regular soluble insulin. Insulin lispro has been chosen due to its rapid onset and short duration of action. The terms inhaled insulin, dry powder insulin, and AI are used interchangeably herein.

### Selection of subjects for clinical evaluation of inhaled insulin

The clinical study described below was carried out with due clinical care in accordance with the declaration of Helsinki, Edinburgh revision, 2000 and conducted in line with the ICH E6 Note for Guidance on Good Clinical Practice. The following criteria were used to select subjects for evaluation of inhaled insulin. Adult male healthy subjects, aged 18 to 45 years, who were non-smokers during the last six months. Selected individuals also had a forced expiratory volume in one second (FEV₁)>80% of predicted volume, and a body mass index of 21 to 27 kg/m². In addition, the selected subjects were willing to refrain from strenuous physical exercise 24 hours prior to the clamp procedure, and had normal (4.4-6.4%) glycosylated haemoglobin (HbA_{1c}).

The following criteria were used to specifically exclude subjects from the study. Those subjects with a history or evidence of lung disease or diabetes were excluded. Subjects with any current or previous significant medical condition or treatment were also excluded. In addition, subjects who had participated in a drug study within the previous 90 days, or who exhibited a clinically significant abnormality on an ECG (electrocardiogram) or routine laboratory blood screen were also specifically excluded from the study.

### Clinical study design

A single cohort, open-label randomized, crossover study of three doses of inhaled insulin was completed. Subjects in the study were assessed during 5 test periods, 3 to 14 days apart, for pharmacodynamic properties by euglycaemic clamp (clamp level 5.0 mmol/L, continuous i.v, insulin infusion of 0.15 mU/kg/min) for 12 hours. After a baseline period of 120 minutes, 12 healthy male volunteers (non-smokers, aged 28.9±5.9 years, BMI 23.5±2.3 kg/m²) received either AI (84, 168 and 294 IU), insulin lispro (IL) (15 IU) or regular soluble insulin (RI) (15 IU). Subjects were trained to inhale through a single step, breath actuated inhaler with a deep, comfortable inhalation.

As the procedure was conducted within the controlled environment of an automated euglycaemic clamp there was no risk of hypoglycaemia to the subject.

Safety and tolerability was assessed by clinical and laboratory evaluations. Blood samples were taken pre-dose and at intervals after dosing to assess the pharmacokinetics of each dose in comparison to insulin lispro and regular soluble insulin. Specifically, three blood samples were taken from each subject for routine safety testing, as described in Table 6. Additionally, up to 21 samples were taken over the course of each treatment day, the volume of which ranged from 2 mL to 3 mL per sample for measurement of glucose, serum insulin and C-peptide. C-peptide is the C chain of insulin, and is endogenous to the human body. Exogenous insulin does not contain the C chain. Thus, by measuring C-peptide in a subject, the level of the subject's endogenous insulin can be determined. The total volume of blood samples taken did not exceed 500 mL in 4 weeks.

TABLE 6
Blood Volumes Collected per Visit

| | | |
|---|---|---|
| Visit | Blood Sample | Blood Volume |

| | | |
|---|---|---|
| Visit 1 | Coagulation tests | 4 mL |
| | Haematology (full safety profile) | 2 mL |
| | Biochemistry (full safety profile) | 2 mL |
| | HbA_{1C} | 2 mL |
| Visits 2, | Haematology (2 mL × 5 visits) | 10 mL |
| 3, 4, 5, 6 | Glucose measurements (2 mL × 5 visits) | 10 mL |
| | Euglycaemic clamp 2 mL/hour (2 mL × 5 | 140 mL |
| | visits × 14 hour) | 264 mL |
| | Test days with study drug insulin (3 mL × 1 | |
| | visit × 15 samples) | 45 mL |
| | C-peptide (7 samples)1* | |
| Visit 7 | Coagulation tests | 4 mL |
| | Biochemistry (full safety profile) | 2 mL |
| | Haematology (full safety profile) | 2 mL |
| Total | | 487 mL |
| Blood | | |

*3 mL includes enough blood for both insulin and C-peptide samples

The full laboratory safety profile included haematology measurements, including haemoglobin count, red cell count, total white cell count, and platelet count. If WBC (white blood cells) results were 10% or greater outside of the normal range, a differential white cell count was performed. Partial Thromboplastin Time (PTT) and International Normalized Ratio (INR) were also determined. In addition, biochemical measurements, including electrolytes (sodium, potassium), creatinine, total protein, bilirubin, alanine transaminase (ALT), gamma GT, alkaline phosphatase, urea concentrations were also measured.

### Study Procedures

### Overall Schedule and Conditions

The schedule for subjects consisted of consent, screening, five within-unit test periods, four washout periods (external to unit) and a final assessment. No strenuous exercise, alcohol or concomitant medication (unless medically indicated) was allowed whilst confined in the unit or during the 24 hours prior to dosing. Subjects were required to fast from 22:00 hours on the preceding day until the end of each test period, and were asked to abstain from drinking coffee at 12 hours prior to dosing until the end of each test period.

### Screening and Initial Assessment

Subjects were screened for entry to the study no more than 21 days prior to visit 2, and entered the study at the point at which they gave informed consent. They were then assigned a subject number and randomized. At this assessment, eligibility was assessed by performing and documenting eligibility according to study inclusion and exclusion criteria; demographics (date of birth, sex, etc); general past medical history; physical examination results, including vital signs, height and weight; ECG results; haematology, biochemistry and urinalysis results; urine drug screen; urine continue test results; HbA_{1c} levels; concomitant medication (prescription only medicines [POM] in the last 14 days and OTC in the last 2 days); adverse events; and baseline lung function test.

The physical examination consisted of a general examination including weight and measurement of height at the initial assessment. Vital signs measurement included supine blood pressure, heart rate, respiration rate and aural temperature, which were measured after 5 minutes rest in the supine position.

Relevant medical and surgical history of each subject was recorded. An indication was also made as to whether any medical condition was ongoing.

As another part of the screening for entry into the study, a 12 lead ECG was measured and evaluated at screening, and thereafter if deemed clinically appropriate.

Urinalysis was also carried out as part of subject screening. The urinalysis involved a semi-quantitative (dipstick) analysis for protein, blood, glucose and ketone.

Urine screen for drugs of abuse includes cannabinoids, barbiturates, amphetamines, benzodiazepines, phenothiazines and cocaine were also carried out as part of subject screening. The urine screen also included testing for cotinine.

Analysis of samples for insulin and C-peptide was conducted by IKFE (Mainz, Germany). Routine safety testing and HbA_{1c} (evaluated on visit 1 only) was determined at FOCUS clinical Drug Development (GmbH, Neuss, Germany). Blood glucose measurements were performed at Profil (Neuss, Germany).

Lung function was measured using a hand held spirometer (Schiller Spirovit SP 200). The actual and expected forced expiratory volume in one second (FEV₁), forced vital capacity (FVC) and mid expiratory flow rate (FEF_{25-75%}) was corrected.

### Inhalation Procedure

The inhalation procedure was practiced with the subjects to familiarize subjects with the procedure and was repeated before each insulin inhalation. Specifically, subjects were trained to inhale through the inhaler with a deep, comfortable inhalation. The investigator removed a capsule from the blister card and placed it in the inhaler device immediately prior to use. Documentation of dose time of inhalation for each dispensation was recorded.

### Test Periods Including Study Drug Administration

The following baseline assessments were performed shortly before connecting the subject to the Biostator to establish euglycaemic glucose clamp: change in physical status since screening and vital signs (supine blood pressure, heart rate, respiration rate and aural temperature); haematology; adverse events since the last visit; and lung function test.

### Procedure for Dose Administrations

The test period started at T=−2 hours, when the subject's blood glucose levels were controlled by means of an automated euglycaemic glucose clamp. This procedure continued from T=−2 hours to T=0.

The subjects were randomized to receive the inhaled insulin. They practiced the inhalation procedure as described in section above during the time T=−2 hours to T=0.

At Time T=0 the subjects received a subcutaneous injection of 15 IU insulin lispro, regular soluble insulin, or a dose of inhaled insulin as indicated by randomization.

When subjects received inhaled insulin the investigator removed a capsule from the blister card (equivalent to 42 IU/capsule) and placed it in the inhaler immediately prior to use. The subject must have been relaxed and breathing normally for at least 5 breaths in order to receive the study drug treatment. The inhaler mouthpiece was placed in the mouth at the end of a normal exhalation. The subject inhaled through the mouth with a deep, comfortable inhalation until he felt that his lungs were full. The subject then held his breath for approximately 5 seconds (by counting slowly to 5).

This procedure was repeated until the correct number of capsules were inhaled to achieve the target insulin dose (see Table 7). Only one breath per capsule was permitted. The time period from the start of the first capsule inhalation (T=0) to the end of the last capsule inhalation was documented.

TABLE 7
Number of capsules for desired dose

| Dose F04-006 IU | No. of Capsules |
|---|---|
| 42 | 1 |
| 84 | 2 |
| 126 | 3 |
| 168 | 4 |
| 210 | 5 |
| 252 | 6 |
| 294 | 7 |

Blood samples were drawn for measurement of insulin levels at times T=−2 hours, −1 hours, 0 (before administration of insulin), 5, 10, 20, 30, 45 minutes, 1.0, 1.5, 2.0, 2.5, 3.0 hours, and then hourly until T=12 hours. Blood samples were drawn for measurement of C-peptide at T=−2, 0, 1, 2, 4, 8 and 12 hours.

A lung function test was performed prior to discharge from the unit. If clinically indicated, ECGs and blood sampling for urea and electrolytes were also carried out.

### Test Period in the Absence of Study Drug Administration

The procedures and assessments for these visits involving test periods in the absence of study drug administration were as described above, except that no study drug was administered. In addition, blood samples for measurement of insulin levels were not collected as described above, but at the following times T=−2 hours, −1 hours, 0 hours (time point at which administration of insulin would have been give for test periods in the presence of study drug administration), then hourly until T=12 hours. Blood samples were drawn for measurement of C-peptide at T=−2, 0, 1, 2, 4, 8 and 12 hours. A lung function test was not conducted on this visit.

### Final Examination

The following final assessments were performed and documented: physical exam and vital signs; haematology, biochemistry and urinalysis results; collection of spontaneously reported adverse events; concomitant medication; ECG if clinically indicated; lung function test; and study completion status.

### Pharmacokinetics

### Sample Handling

The handling of samples for insulin and C-peptide measurements was carried out as follows. After collection, blood samples were allowed to clot in tubes at room temperature for at least 30 minutes but not longer than 1 hour. Following centrifugation at room temperature (2000 g for 10 minutes) the serum was stored frozen in, screw-capped polypropylene tubes. Samples from each individual subject were stored as a package for that subject. Insulin levels for each subject were measured using the Coat-A-Coat™ Insulin RIA KIT (Diagnostic Products Corporation TK1N2), and C-peptide levels were determined using the Human C-peptide RIA (radio-Immuno assay) Kit (Linco Research Inc. HCP 20K). Established procedures, known in the art, were applied for characterizing concentration-time profiles of insulin and C-peptide in serum.

### Prescribed Unit Dose of Study Drugs

The drugs used in the study were: inhaled insulin powder (equivalent to 42 IU/capsule recombinant human insulin); insulin lispro and regular soluble insulin (1.5 mL cartridges each providing 100 IU/mL of which 0.150 mL of was administered). Insulin for inhalation was manufactured and provided by Applicant as capsules containing the equivalent of 42 IU/capsule recombinant human insulin powdered drug substance. Inhaled insulin was not stored above 25° C.

### Results

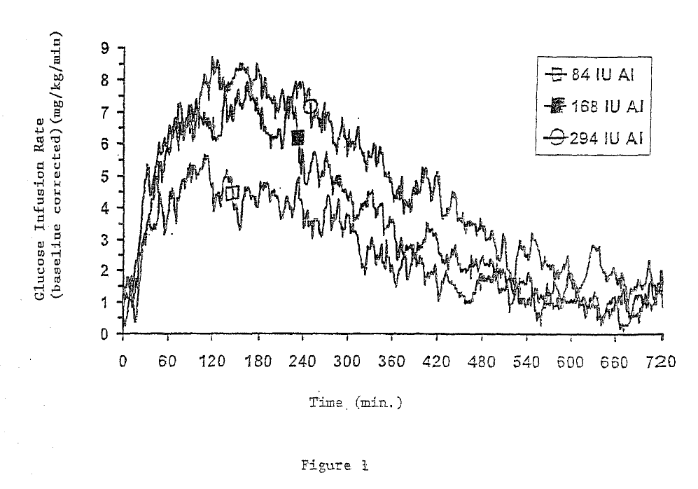

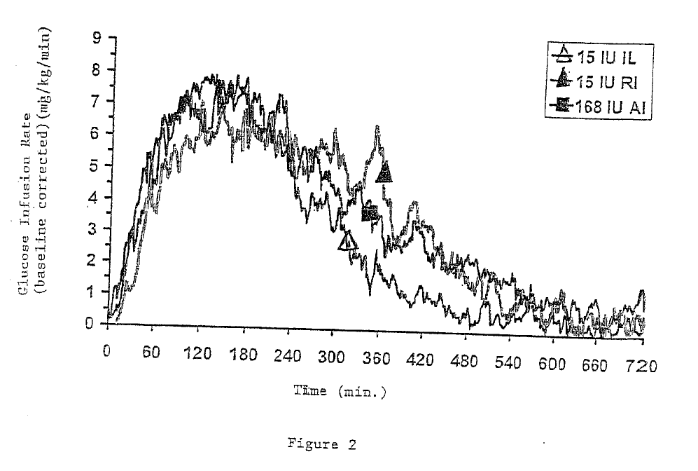

As shown in FIG. 1, the glucose infusion rate in those subjects receiving inhaled insulin was dose dependent. In addition, FIG. 2, shows the glucose infusion rate in subjects receiving 168 IU of inhaled insulin, insulin lispro, or regular soluble insulin. The pharmacodynamic properties of 168 IU inhaled insulin were comparable to those of insulin lispro and regular soluble insulin.

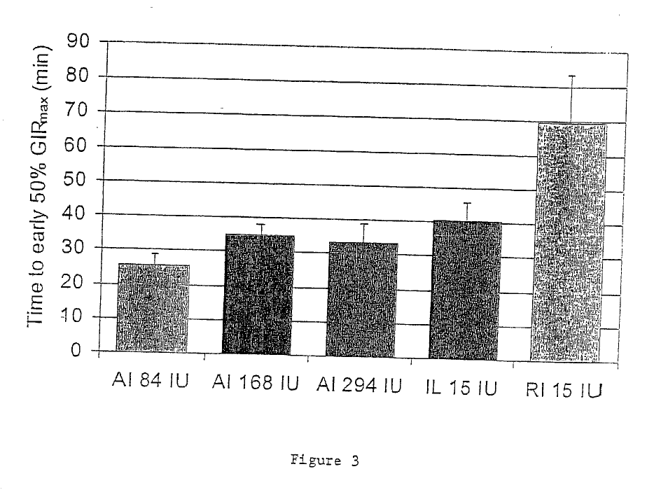

The onset actions of inhaled insulin, insulin lispro, and regular soluble insulin were also evaluated for those subjects involved in the study described above. The onset action, described as the T_{max50%} (in minutes), was calculated for each subject. As shown in FIG. 3, the T_{max50%} was lower for all doses of the inhaled insulin preparations, compared to the insulin lispro and regular soluble insulin. Specifically, AI showed a faster onset of action compared with subcutaneous insulin formulations lispro (IL) and regular soluble insulin (RI) (early Tmax 50%[min]: 29 (84 IU), 35 (168 IU), 33 (294 IU), 41 (IL) and 70 (RI) [p<0.01 for AI (all doses) compared to RI]). These results therefore show that the inhaled insulin preparations had a faster onset of action.

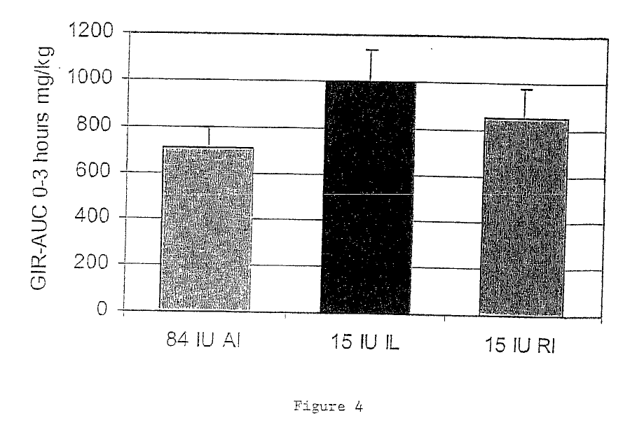

In addition, the GIR-AUC_{0-3 hours} was assessed for each subject in the study. In the first three hours after drug administration (a typical meal related period), the 84 IU dose of inhaled insulin gave a GIR-AUC_{0-3 hours} closest to regular insulin, as shown in FIG. 4.

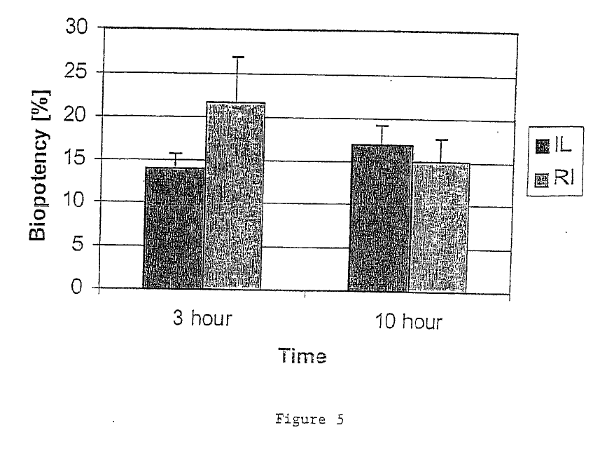

The biopotency of 84 IU inhaled insulin was compared to the biopotency of insulin lispro and regular soluble insulin. As shown in FIG. 5, for the first three hours after drug administration, the biopotency of 84 IU of inhaled insulin was 22% relative to regular soluble insulin, and 14% relative to insulin lispro. Ten hours after administration, the biopotency of inhaled insulin (84 IU) was 16% compared to the biopotency of regular soluble insulin, and 18% compared to insulin lispro.

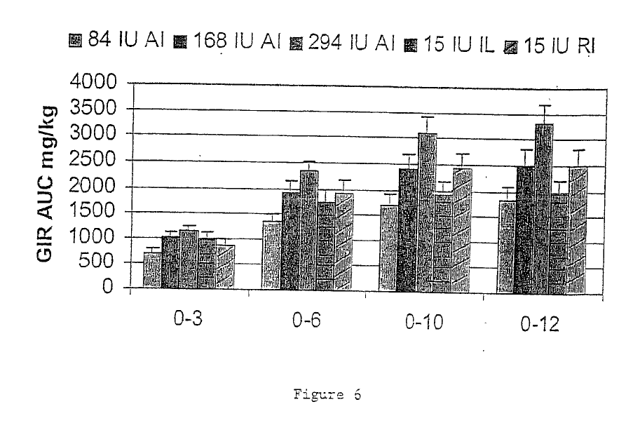

The GIR-AUC, evaluated as a function of time was also calculated for each formulation, as shown in FIG. 6.

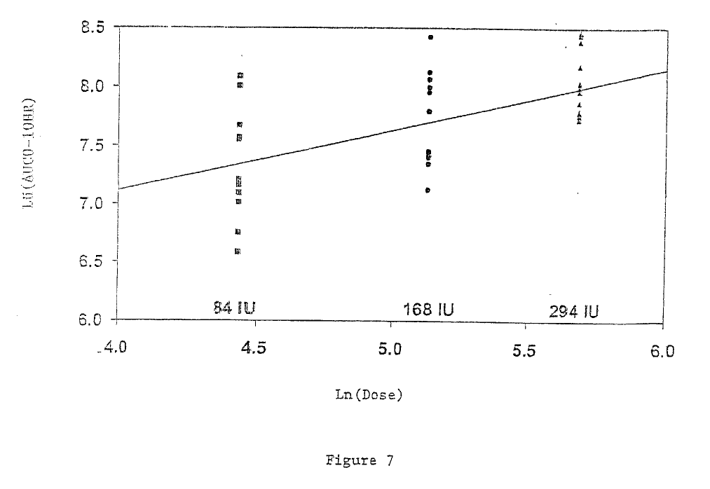

The effects of the three different concentrations of inhaled insulin (natural log of 84 IU, 168 IU, and 294 IU) were also evaluated for their effect on glucose infusion rates (natural log of the GIR-AUC_{0-10 hours}) for each subject over a period of time from zero to ten hours after drug administration. This analysis, as shown in FIG. 7, revealed a linear dose response rate over the range of inhaled insulin concentrations studied.

Finally, the inter-subject variability of the pharmacodynamic properties of the drugs administered in this study were examined, by calculating the coefficient of variation for each drug administered. As shown in Table 8, the inter-subject variability, based on AUC0-10 hours following oral inhalation of insulin showed a similar coefficient of variation (CV) to insulin administered by subcutaneous injection. In addition, the intra-subject CV for all doses of inhaled insulin was estimated to be 20% at AUCO_{0-3 hours}, and 19% at AUC_{0-10 hours}. These estimates were obtained using a linear mixed model on log transformed AUC data, with the subject as a random effect and inhaled insulin dose as a fixed effect.

TABLE 8
Inter-subject Variability of Drugs

| | Inter-subject Coefficient of Variation |
|---|---|
| Drug Administered | (%) |
| 15 IU insulin lispro | 44 |
| 15 IU regular soluble insulin | 45 |
| 84 IU inhaled insulin | 48 |
| 168 IU inhaled insulin | 41 |
| 294 IU inhaled insulin | 35 |

While this invention has been particularly shown and described with references to preferred embodiments thereof, it will be understood by those skilled in the art that various changes in form and details may be made therein without departing from the scope of the invention encompassed by the appended claims.

What is claimed is:

1. A formulation having particles comprising, by weight, 60% DPPC, 30% insulin and 10% sodium citrate.

2. A formulation having particles comprising, by weight, 40% DPPC, 50% insulin and 10% sodium citrate.

3. A formulation having particles comprising, by weight, 40% to 60% DPPC, 30% to 50% insulin and 10% sodium citrate.

4. A formulation having particles comprising, by weight, 80% DPPC, 10% insulin and 10% sodium citrate.

5. A formulation having particles comprising, by weight, 75% DPPC, 15% insulin and 10% sodium citrate.

6. A formulation having particles comprising, by weight, 75% to 80% DPPC, 10% to 15% insulin and 10% sodium citrate.

7. The formulation of claim 6, wherein the particles comprise a mass of from about 1.5 mg to about 20 mg of insulin.

8. The formulation of claim 6, wherein the particles comprise a mass of about 1.5 mg of insulin per receptacle.

9. The formulation of claim 6, wherein the particles comprise a mass of about 5 mg of insulin per receptacle.

10. The formulation of claim 6, wherein the particles comprise a dosage of insulin between about 42 IU and about 540 IU.

11. The formulation of claim 10, wherein the particles comprise a dosage of insulin of about 42 IU.

12. The formulation of claim 10, wherein the particles comprise a dosage of insulin of between about 84 IU and about 294 IU.

13. The formulation of claim 6, wherein the particles have a tap density less than about 0.4 g/cm³.

14. The formulation of claim 13, wherein the particles have a tap density less than about 0.1 g/cm³.

15. The formulation of claim 6, wherein the particles have a median geometric diameter of from about 5 micrometers to about 30 micrometers.

16. The formulation of claim 15, wherein the particles have a median geometric diameter of from about 7 micrometers to about 8 micrometers.

17. The formulation of claim 6, wherein the particles have an aerodynamic diameter of from about 1 micrometer to about 5 micrometers.

18. The formulation of claim 17, wherein the particles have an aerodynamic diameter of from about 1 micrometer to about 3 micrometers.

19. The formulation of claim 17, wherein the particles have an aerodynamic diameter of from about 3 micrometers to about 5 micrometers.

20. The formulation of claim 6, wherein the particles further comprise an amino acid.

21. The formulation of claim 20, wherein the amino acid is leucine, isoleucine, alanine, valine, phenylalanine or any combination thereof.

22. A method for treating a human patient in need of insulin comprising administering pulmonarily to the respiratory tract of a patient in need of treatment, an effective amount of particles comprising by weight, 60% DPPC, 30% insulin and 10% sodium citrate, wherein release of the insulin is rapid.

23. A method for treating a human patient in need of insulin comprising administering pulmonarily to the respiratory tract of a patient in need of treatment, an effective amount of particles comprising by weight, 40% DPPC, 50% insulin and 10% sodium citrate, wherein release of the insulin is rapid.

24. A method for treating a human patient in need of insulin comprising administering pulmonarily to the respiratory tract of a patient in need of treatment, an effective amount of particles comprising by weight, 40% to 60% DPPC, 30% to 50% insulin and 10% sodium citrate, wherein release of the insulin is rapid.

25. A method for treating a human patient in need of insulin comprising administering pulmonarily to the respiratory tract of a patient in need of treatment, an effective amount of particles comprising by weight, 80% DPPC, 10% insulin and 10% sodium citrate, wherein release of the insulin is rapid.

26. A method for treating a human patient in need of insulin comprising administering pulmonarily to the respiratory tract of a patient in need of treatment, an effective amount of particles comprising by weight, 75% DPPC, 15% insulin and 10% sodium citrate, wherein release of the insulin is rapid.

27. A method for treating a human patient in need of insulin comprising administering pulmonarily to the respiratory tract of a patient in need of treatment, an effective amount of particles comprising by weight, 75% to 80% DPPC, 10% to 15% insulin and 10% sodium citrate, wherein release of the insulin is rapid.

28. The method of claim 27, wherein the patient in need of treatment has diabetes mellitus.

29. The method of claim 27, wherein the particles have a mass of from about 1.5 mg to about 20 mg of insulin.

30. The method of claim 27, wherein the particles comprise a mass of about 1.5 mg of insulin per receptacle.

31. The method of claim 27, wherein the particles comprise a mass of about 5 mg of insulin per receptacle.

32. The method of claim 27, wherein the particles comprise a dosage of insulin of between about 42 IU and about 540 IU.

33. The method of claim 32, wherein the particles comprises a dosage of insulin of about 42 IU.

34. The method of claim 32, wherein the particles comprise a dosage of insulin of between about 84 IU and about 294 IU.

35. The method of claim 27, wherein the particles have a tap density less than about 0.4 g/cm³.

36. The method of claim 35, wherein the particles have a tap density less than about 0.1 g/cm³.

37. The method of claim 27, wherein the particles have a median geometric diameter of from about 5 micrometers to about 30 micrometers.

38. The method of claim 37, wherein the particles have a median geometric diameter from about 7 micrometers to about 8 micrometers.

39. The method of claim 27, wherein the particles have an aerodynamic diameter of from about 1 micrometer to about 5 micrometers.

40. The method of claim 39, wherein the particles have an aerodynamic diameter of from about 1 micrometers to about 3 micrometers.

41. The method of claim 39, wherein the particles have an aerodynamic diameter of from about 3 micrometers to about 5 micrometers.

42. The method of claim 27, wherein administering the particles pulmonarily includes delivery of the particles to the deep lung.

43. The method of claim 27, wherein administering the particles pulmonarily includes delivery of the particles to the central airways.

44. The method of claim 27, wherein administering the particles pulmonarily includes delivery of the particles to the upper airways.

45. The method of claim 27, wherein the particles further comprise an amino acid.

46. The method of claim 45, wherein the amino acid is leucine, isoleucine, alanine, valine, phenylalanine or any combination thereof.

47. A method of delivering an effective amount of insulin to the pulmonary system, comprising:
a) providing a mass of particles comprising by weight, 60% DPPC, 30% insulin and 10% sodium citrate; and
b) administering via simultaneous dispersion and inhalation the particles, from a receptacle having the mass of the particles, to a human subject's respiratory tract, wherein release of the insulin is rapid.

48. A method of delivering an effective amount of insulin to the pulmonary system, comprising:
a) providing a mass of particles comprising by weight, 40% DPPC, 50% insulin and 10% sodium citrate; and
b) administering via simultaneous dispersion and inhalation the particles, from a receptacle having the mass of the particles, to a human subject's respiratory tract, wherein release of the insulin is rapid.

49. A method of delivering an effective amount of insulin to the pulmonary system, comprising:
a) providing a mass of particles comprising by weight, 40% to 60% DPPC, 30% to 50% insulin and 10% sodium citrate; and
b) administering via simultaneous dispersion and inhalation the particles, from a receptacle having the mass of the particles, to a human subject's respiratory tract, wherein release of the insulin is rapid.

50. A method of delivering an effective amount of insulin to the pulmonary system, comprising:
a) providing a mass of particles comprising by weight, 80% DPPC, 10% insulin and 10% sodium citrate; and
b) administering via simultaneous dispersion and inhalation the particles, from a receptacle having the mass of the particles, to a human subject's respiratory tract, wherein release of the insulin is rapid.

51. A method of delivering an effective amount of insulin to the pulmonary system, comprising:
a) providing a mass of particles comprising by weight, 75% DPPC, 15% insulin and 10% sodium citrate; and
b) administering via simultaneous dispersion and inhalation the particles, from a receptacle having the mass of the particles, to a human subject's respiratory tract, wherein release of the insulin is rapid.

52. A method of delivering an effective amount of insulin to the pulmonary system, comprising:
a) providing a mass of particles comprising by weight, 75% to 80% DPPC, 10% to 15% insulin and 10% sodium citrate; and
b) administering via simultaneous dispersion and inhalation the particles, from a receptacle having the mass of the particles, to a human subject's respiratory tract, wherein release of the insulin is rapid.

53. The method of claim 52, wherein the particles comprise a mass of from about 1.5 mg to about 20 mg of insulin.

54. The method of claim 52, wherein the particles comprise a mass of about 1.5 mg of insulin per receptacle.

55. The method of claim 52, wherein the particles comprise a mass of about 5 mg of insulin per receptacle.

56. The method of claim 52, wherein the particles comprise a dosage of insulin of between about 42 IU and about 540 IU.

57. The method of claim 56, wherein the particles comprise a dosage of insulin of about 42 IU.

58. The method of claim 56, wherein the particles comprise a dosage of insulin of between about 84 IU and about 294 IU.

59. The method of claim 52, wherein the particles have a tap density less than about 0.4 g/cm³.

60. The method of claim 59, wherein the particles have a tap density less than about 0.1 g/cm³.

61. The method of claim 52, wherein the particles have a median geometric diameter of from about 5 micrometers to about 30 micrometers.

62. The method of claim 61, wherein the particles have a median geometric diameter of from about 7 micrometers to about 8 micrometers.

63. The method of claim 52, wherein the particles have an aerodynamic diameter of from about 1 micrometer to about 5 micrometers.

64. The method of claim 63, wherein the particles have an aerodynamic diameter of from about 1 micrometer to about 3 micrometers.

65. The method of claim 63, wherein the particles have an aerodynamic diameter of from about 3 micrometers to about 5 micrometers.

66. The method of claim 52, wherein delivery to the pulmonary system includes delivery to the deep lung.

67. The method of claim 52, wherein delivery to the pulmonary system includes delivery to the central airways.

68. The method of claim 52, wherein delivery to the pulmonary system includes delivery to the upper airways.

69. The method of claim 52, wherein the particles further comprise an amino acid.

70. The method of claim 69, wherein the amino acid is leucine, isoleucine, alanine, valine, phenylalanine or any combination thereof.

71. The formulation of claim 6, wherein the particles further comprise a low transition temperature phospholipid.

72. The method of claim 27, wherein the particles further comprise a low transition temperature phospholipid.

73. A kit for administration of insulin comprising two or more receptacles, wherein said receptacles comprise unit dosages selected from the group consisting of
a) particles comprising, by weight, 60% DPPC, 30% insulin and 10% sodium citrate;
b) particles comprising, by weight, 40% DPPC, 50% insulin and 10% sodium citrate;
c) particles comprising, by weight, 40% to 60% DPPC, 30% to 50% insulin and 10% sodium citrate;
d) particles comprising, by weight, 75% DPPC, 15% insulin and 10% sodium citrate;
e) particles comprising, by weight, 80% DPPC, 10% insulin and 10% sodium citrate; and
f) particles comprising, by weight, 75% to 80% DPPC, 10% to 15% insulin and 10% sodium citrate.

74. The kit of claim 73, wherein said kit further comprises instructions for use of said two or more receptacles.

75. The kit of claim 73, wherein one or more receptacles comprise unit dosages of particles comprising, by weight, 40% to 60% DPPC, 30% to 50% insulin and 10% sodium citrate and wherein one or more receptacles comprise unit dosages of particles comprising, by weight, 75% to 80% DPPC, 10% to 15% insulin and 10% sodium citrate.

76. The kit of claim 73, wherein one or more receptacles comprise unit dosages of particles comprising, by weight, 60% DPPC, 30% insulin and 10% sodium citrate and wherein one or more receptacles comprise unit dosages of particles comprising, by weight, 80% DPPC, 10% insulin and 10% sodium citrate.

77. A formulation having particles comprising, by weight, 60% DPPC, 30% insulin and 10% sodium citrate, wherein the method of preparing said formulation comprises
a) preparing a solution of DPPC;
b) preparing a solution of insulin and sodium citrate;
c) heating the solutions of steps a) and b) to a temperature of 50° C.;
d) combining the heated solutions of step c) such that the total solute concentration is greater than 3 grams per liter; and
e) spray drying the solution formed in step d) solution to form particles.

78. The method of claim 77, wherein in step d) said solute concentration is 15 grams per liter.

79. A kit comprising at least two receptacles each receptacle containing a different amount of dry powder insulin suitable for inhalation.
